(19) United States
(12) Patent Application Publication
(10) Pub. No.: US 2012/0203128 A1
(43) Pub. Date: Aug. 9, 2012

(54) RESPIRATORY RATE DETECTION DEVICE, SYSTEM AND METHOD

(76) Inventors: Jeffrey Alexander Levison, Mesa, AZ (US); Michael Brian Frieswyk, Scottsdale, AZ (US)

(21) Appl. No.: 13/369,160

(22) Filed: Feb. 8, 2012

Related U.S. Application Data
(60) Provisional application No. 61/440,733, filed on Feb. 8, 2011.
(60) Provisional application No. 61/530,910, filed on Sep. 2, 2011.
(60) Provisional application No. 61/548,167, filed on Oct. 17, 2011.

(51) Int. Cl. A61B 5/08

(52) U.S. Cl. 600/537; 600/529

(57) ABSTRACT

A respiration rate measurement device comprising a tubular housing configured to be disposed over a nose and mouth on a face of a subject. The tubular housing comprises a proximal end configured to communicate with the nose and mouth of the subject and receive a transient pressure event from the nose and mouth and a distal end that opens to ambient atmosphere. The tubular housing is configured to guide a flow of air, generated from the transient pressure event, between the proximal end and the distal end. The respiration rate measurement device further comprises a sensor disposed within the housing. The sensor is configured to detect a respiration event by monitoring the flow of air within the tubular housing.

### CROSS-REFERENCES TO RELATED APPLICATIONS

This non-provisional application claims priority to, and the benefit of, U.S. Provisional Pat. Application Ser. No. 61/440,733, filed Feb. 8, 2011, entitled “Respiratory Rate Detection System And Method Of Using Same,” the entire contents of which is hereby incorporated by reference, and U.S. Provisional Pat. Application Ser. No. 61/530,910, filed Sep. 2, 2011, entitled “Respiratory Rate Detection System And Method Of Using Same,” the entire contents of which is hereby incorporated by reference, and U.S. Provisional Pat. Application Ser. No. 61/548,167, filed Oct. 17, 2011, entitled “Respiratory Rate Detection System And Method Of Using Same,” the entire contents of which is hereby incorporated by reference.

### TECHNICAL FIELD

The Applicants' invention relates to a system to measure respiratory rate and more particularly to an apparatus that may be used to continuously monitor and display the respiratory rate of a patient in various health care scenarios and issue alerts based on a threshold respiratory rate or changes in the measured respiratory rate.

### BACKGROUND ART

Five vital signs must be measured when triaging a patient: heart rate, temperature, blood pressure, pulse oximetry and respiratory rate. The heart rate, temperature, blood pressure, and pulse oximetry can be accurately measured in an easy and economical fashion using devices and techniques known in the art.

By contrast, techniques to measure respiratory rate often introduce significant measurement errors into the resultant value due to the subjective nature in which the data is obtained. Respiratory rate, also known as breathing rate, ventilation rate, or respiration rate, is the number of breaths a person takes within a specific amount of time, generally a minute. A breath is defined as either an inhalation event or an exhalation event. Respiratory rate generally varies by age. The typical respiratory rate for an adult at rest is between 12 and 20 breathes per minute (i.e., 12-20 inhalations per minute or 12-20 exhalations per minute).

Respiratory rate is an important health indicator. Studies have shown a direct correlation between an elevated respiratory rate and impending cardiopulmonary collapse and death. An increased respiratory count is usually the result of a serious medical condition such as myocardial infarction, pulmonary embolus, metabolic acidosis, pneumonia, or ARDS (acute respiratory distress syndrome).

The standard technique for measuring respiratory rate is by the manual counting of breaths by medical personnel. The counting is accomplished by observing the number of times the stomach or chest rises in a short period of time and then extrapolating to a full minute. For example, counting the number of breaths over a 30 second period and multiplying the count by 2 will give the number of breaths per minute.

The respiratory rate results obtained from using the standard technique is subject to error for numerous reasons. First, many ancillary medical personnel such as medical assistants or med techs may have not learned how to correctly obtain this data, nor do they have the clinical skills to know when a patient may be in respiratory distress. Second, the measurement represents a small snapshot in time, which does not necessarily reflect the respiratory condition after the measurement and is not effective in detecting a respiratory condition that is deteriorating over time. During times of heavy patient volume, wait times can be extensive, and a patient may not be seen again after his initial triage for an extended period of time. Finally, even with properly trained personnel, the detection of a breath is often subjectively determined. Observing the small rise and fall of the chest or stomach of a patient may be difficult in many instances. Furthermore, the misidentification of a single breath during a 30 second measurement period will result in a deviation of 10% or greater from the actual respiratory rate. Erroneously taken respiratory rates often result in the mismanagement of the patient and can frequently lead to extremely adverse and catastrophic outcomes.

Policy and protocol for medical institutions can be based around the results of a respiratory counter capable of removing the subjective (human) factor from the respiratory rate equation and allowing for the ongoing monitoring of respiratory rate over time. A rate above a certain number would be reported immediately to the provider on duty, allowing the provider to appropriately respond to the situation and treat the patient expeditiously, rather than waiting for the cardiopulmonary arrest that might occur due to the misrepresentation of a patient's respiratory status. Accordingly, it would be an advance in the state of the art to provide a device that is capable of measuring respiratory rate directly and with high accuracy, that is inexpensive and reusable, and that could be easily incorporated into the vital stand apparatus that is commonly used in various medical settings today, or exists as a stand-alone device. Such a device would improve patient care, resulting in less morbidity and less mortality associated with visits to the primary care doctor, urgent care facilities, and emergency rooms.

### SUMMARY OF THE INVENTION

A respiration rate measurement device is presented. The respiration rate measurement device comprises a tubular housing configured to be disposed over a nose and mouth on a face of a subject. The tubular housing comprises a proximal end configured to communicate with the nose and mouth of the subject and receive a transient pressure event from the nose and mouth and a distal end that opens to ambient atmosphere. The tubular housing is configured to guide a flow of air, generated from the transient pressure event, between the proximal end and the distal end. The respiration rate measurement device further comprises a sensor disposed within the housing. The sensor is configured to detect a respiration event by monitoring the flow of air within the tubular housing.

A respiratory rate detection system is further presented. The respiration rate detection system comprises a tubular housing configured to be disposed over a nose and mouth on a face of a subject. The tubular housing comprises a proximal end configured to communicate with the nose and mouth of the subject and receive a transient pressure event from the nose and mouth and a distal end that opens to ambient atmosphere. The tubular housing is further configured to guide a flow of air, generated from the transient pressure event, between the proximal end and the distal end. The respiration rate detection system further comprises a sensor disposed within the housing. The sensor is configured to detect a respiration event by monitoring the flow of air within the tubular housing. The respiration rate detection system further comprises a processor and a computer readable medium comprising computer readable program code disposed therein to determine a respiration rate based on a plurality of detected events registered by the sensor. The computer readable program code comprises a series of computer readable program steps to effect initiating a timer to trigger at a periodic and predetermined time interval, receiving respiration event data from the sensor corresponding to the plurality of respiration events from the sensor, filtering the respiration rate data to remove background noise and to identify at least one individual respiration cycle, incrementing a value of a breath count variable for each of the individual respiration cycle, and calculating the respiration rate upon the triggering of the timer.

### BRIEF DESCRIPTION OF THE DRAWINGS

The invention will be more fully understood by referring to the following Detailed Description of Specific Embodiments in conjunction with the Drawings, of which:

FIG. 1 is one embodiment of Applicants' respiratory rate detection system attached to a patient and configured to continuously monitor respiratory rate;

FIG. 2 is a side view of the mask portion of the respiratory rate detection system of FIG. 1;

FIGS. 3(a) and 3(b) show an embodiment of Applicants' respiratory rate detection system using a microphone to detect breaths and the details of the microphone;

FIG. 4 is an embodiment of Applicants' respiratory rate detection system using a optical flow meter to detect breaths;

FIGS. 5(a)-(c) show an embodiment of Applicants' respiratory rate detection system using a system of vanes to detect breaths;

FIGS. 6(a) to 6(c) is an embodiment of Applicants' respiratory rate detection system using a contact panel to detect breaths;

FIG. 7 is an embodiment of Applicants' respiratory rate detection system using a CO₂ detector to detect breaths;

FIG. 8 is an embodiment of Applicants' respiratory rate detection system using a moisture or temperature detector to detect breaths;

FIG. 9 is a graph showing the output of a respiratory rate detection system;

FIG. 10 shows a side view of one embodiment of Applicants' respiratory rate detection system configured to determine the current respiratory rate of a patient;

FIGS. 11(a)-11(c) show various embodiments of Applicants' respiratory rate detection system configured to detect respiratory rate by contacting the patient's neck;

FIG. 12 is a flowchart showing one exemplary method of determining the current respiratory rate of a patient by using Applicants' respiratory rate detection system;

FIG. 13 is an embodiment of Applicants' respiratory rate detection system using a thermal anemometer to detect breaths;

FIG. 14 is a three dimensional view of one embodiment of Applicants' respiratory rate detection system in use;

FIG. 15 is an additional three dimensional view of one embodiment of Applicants' respiratory rate detection system;

FIG. 16(a) is a three dimensional view of one embodiment of Applicants' respiratory rate detection system with a sanitary liner;

FIGS. 16(b) and 16(c) are three dimensional views of different embodiments of sanitary liners to be used with Applicants' respiratory rate detection system;

FIGS. 17(a) and 17(b) are front, right, top perspective views of different embodiments of Applicants' respiratory rate detection unit;

FIGS. 18(a) and 18(b) are right side elevational views of different embodiments of Applicants' respiratory rate detection unit, the left side elevational views thereof being a mirror image of the right side shown;

FIGS. 19(a) and 19(b) are front elevational views of different embodiments of Applicants' respiratory rate detection unit;

FIGS. 20(a) and 20(b) are rear elevational views of different embodiments of Applicants' respiratory rate detection unit;

FIG. 21(a) is a top plan view of one embodiment of Applicants' respiratory rate detection unit;

FIG. 21(b) is a cross sectional view of the tubular housing of Applicants' respiratory rate detection unit along section line B-B of FIG. 21(a);

FIG. 22(a) is a top plan view of one embodiment of Applicants' respiratory rate detection unit having a liner;

FIG. 22(b) is a cross sectional view of the tubular housing of Applicants' respiratory rate detection unit having a line along section line B-B of FIG. 22(a); and

FIGS. 23(a) and 23(b) are bottom plan views of different embodiments of Applicants' respiratory rate detection unit.

### DETAILED DESCRIPTION OF SPECIFIC EMBODIMENTS

Referring to the foregoing paragraphs, this invention is described in preferred embodiments in the following description with reference to the Figures, in which like numerals represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of the invention may be combined in any suitable manner in one or more embodiments. In the above description, numerous specific details are recited to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

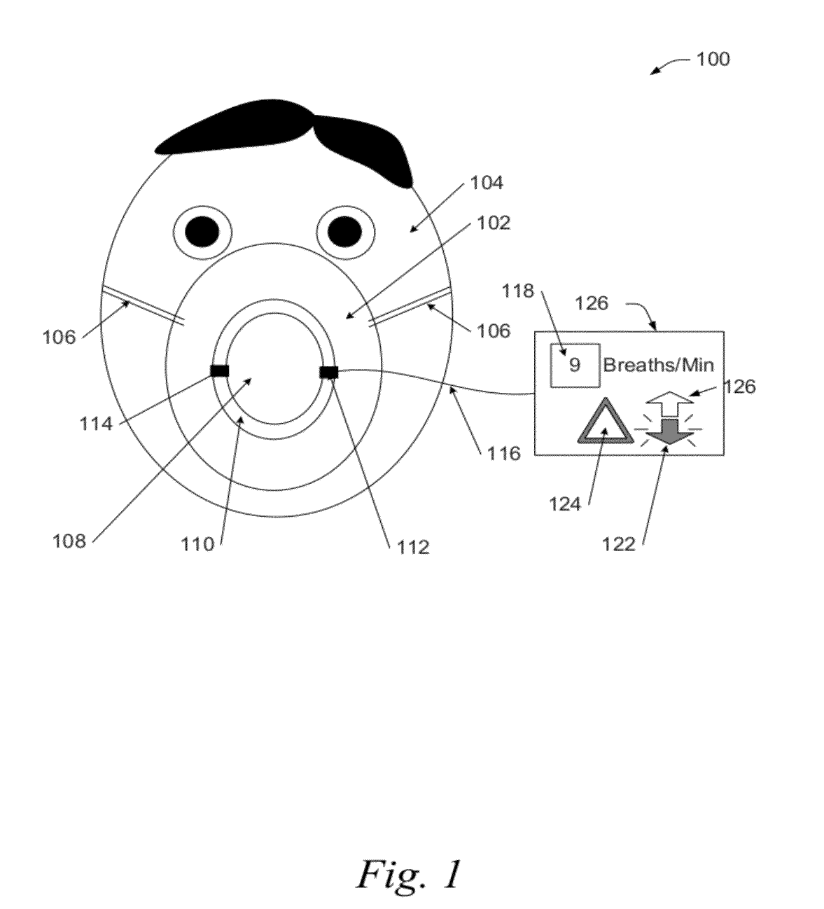

Referring to FIG. 1, an exemplary embodiment of Applicants' respiratory rate detection system 100 is depicted. A mask 102 is held over the nose and mouth of a subject 104 by a strap 106. In various embodiments, the subject 1402 may be a human or a domesticated animal, such as without limitation a dog, cat, or horse. An opening 108 is formed in the mask 102. The air exhaled from the subject's 104 nose and/or mouth is channeled by the mask 102 to flow out of the opening 108. Similarly, as the patient inhales, air is pulled in through the opening 108 before it enters the patient's nose and/or mouth. As such, the opening 108 concentrates the flow of air as the subject 104 breathes. The opening 108 also allows measurement of the air flow and related properties without any appreciable interference or obstruction of the air supply to the patient. Unlike systems that fully cover the mouth and/or nose using, among other devices, an oxygen mask, or systems that funnel the entire flow of air into a detection unit, the mask 102 with opening 108 allows the subject 104 to breathe normally during respiratory rate detection.

In one embodiment, a detection ring 110 is disposed within the opening 108 and circumscribes the interior surface of the opening 108. In another embodiment, a detection ring 110 is aligned with the opening 108 and disposed above the opening 108 (i.e., on the side of the mask 102 opposite the patient's face) such that the flow of air passes through the detection ring 110.

In one embodiment, the detection ring 110 is releasably attached to mask 102. In this embodiment, the mask 102 is disposable. As such, the detection ring 110 may be attached to a new mask for each subject 104 and the old mask may be discarded.

In one embodiment, the detection ring 110 has sensors 112 and 114. The sensors 112 and 114 are in communication with the control unit 126. In one embodiment, the sensors 112 and 114 are in communication with control unit 126 by a wire 116. In other embodiments, the sensors 112 and 114 are in communication with control unit 126 by a wireless signal. In one embodiment, the information is transmitted through a wireless connection that conforms with a wireless standard, such as without limitation Bluetooth (IEEE 802.15.1 and later implementations), Wi-Fi (IEEE 802.11), irDA, implementations of IEEE 802.15.4 (ex., ZigBee), and Z-Wave. In one embodiment, the detection ring 110 contains a battery, a processor, and an antenna to convert and transmit data from sensors 112 and 114 to control unit 126. In one embodiment, the processor in detection ring 110 processes the data from sensors 112 and 114 to produce a respiratory rate value to be communicated to and displayed on control unit 126.

In different embodiments, the sensors disposed on the mask 102 transmit one or a combination of the following to the control unit 126: sensor diagnostics, ambient temperature, oxygen content of the patient's breath, alcohol content of the patient's breath, and sensor usage information, such as total use time, time to maintenance (i.e., when the sensor should be cleaned and/or calibrated), or time to replace (i.e., when the sensor should be replaced). In one embodiment, the sensor includes a unit ID. In one embodiment, the unit ID is a 128-bit universally unique identifier (UUID) that is capable of uniquely identifying a specific sensor. In one embodiment, the UUID is associated with a patient and/or an attendant.

In one embodiment, control unit 126 includes a digital display 118 used to continuously display the measured respiratory rate. In one embodiment, the digital display 118 is a multifunction display capable of displaying various types of information. In one embodiment, the control unit 126 includes an alert indicator to alert medical personnel of a potentially dangerous respiratory condition, including a respiratory rate above a threshold level, a respiratory rate below a threshold level or a change in the respiratory rate between two time periods above or below a threshold level. In one embodiment, arrow 120 illuminates to indicate a respiratory rate above a threshold level or a respiratory rate that is trending up in a dangerous manner. In one embodiment, the arrow 122 illuminates to indicate a respiratory rate below a threshold level or a respiratory rate that is trending down in a dangerous manner. In one embodiment, the alert indicator 124 illuminates to indicate a dangerous respiratory rate condition.

In different embodiments, the control unit 126 displays one or more of the: breath rate, date/time, patient ID, attendant ID, ambient temperature, real time sensor data, calibration information, oxygen levels of the patient's breath, alcohol levels of the patient's breath, carbon dioxide levels of the patient's breath, temperature of the patent's breath, temperature difference from ambient temperature, the sensor unit ID, the control unit ID, and sensor usage information.

In different embodiments, the control unit 126 may be a multi-function mobile device, such as a smart phone (i.e., android, iPhone, or Blackberry), a vital stand apparatus typically used in hospitals and health care centers, or a custom display device configured to function solely with the detection ring 110.

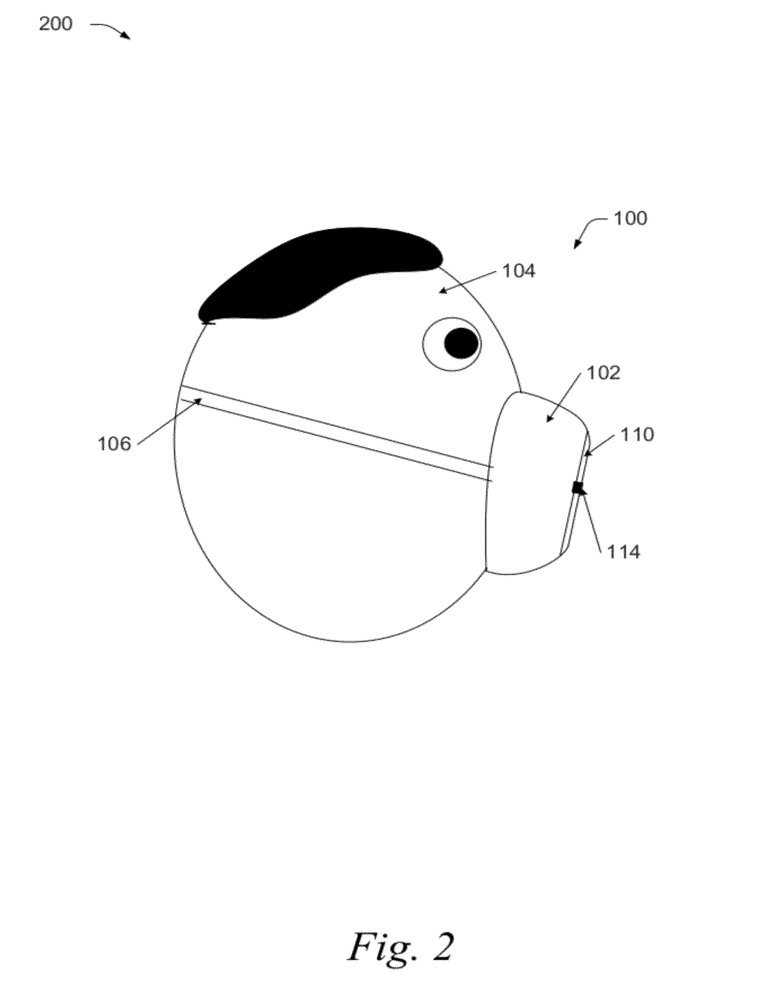

Referring to FIG. 2, a side view of the embodiment in FIG. 1 is depicted. A mask 102 is disposed over the nose and mouth of a subject 104. The mask is held in place by a strap 106. The detector ring 110 is disposed on the mask 102. A sensor 114 is disposed on the detector ring 110.

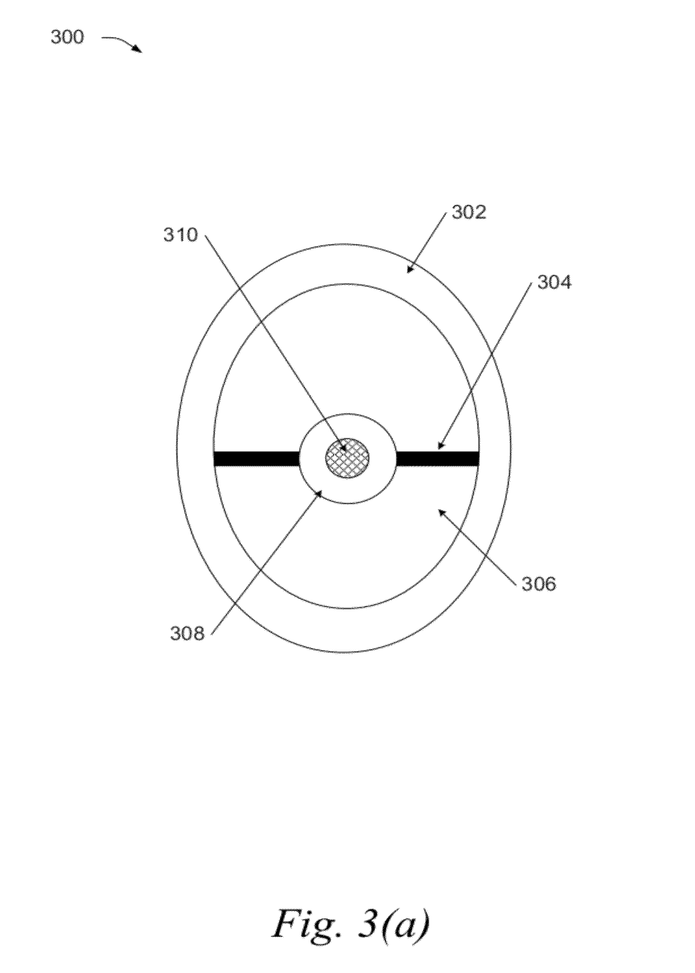

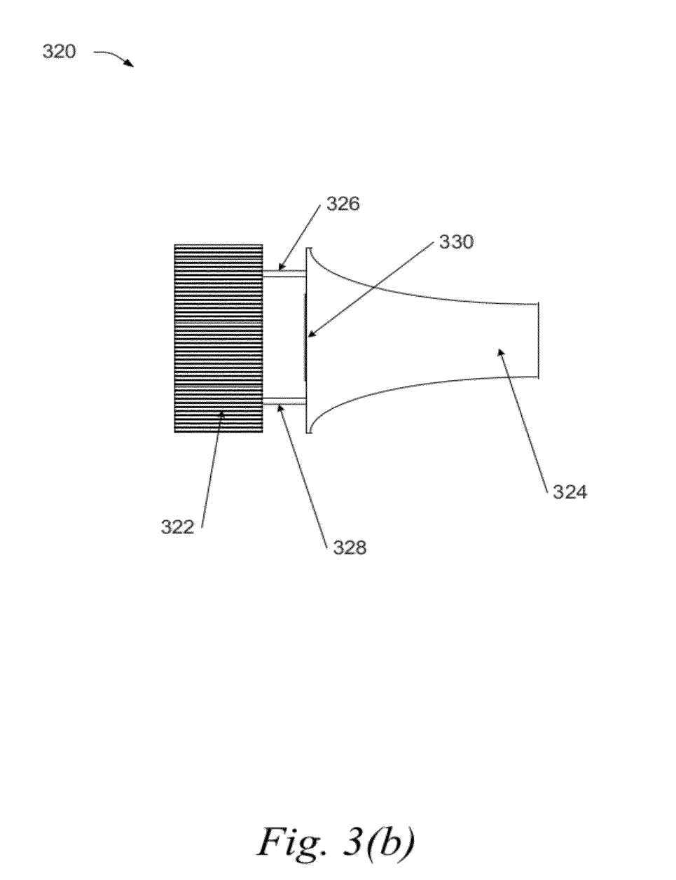

Referring to FIG. 3(a), one embodiment of a respiratory rate detection ring assembly 300 using a microphone for use in Applicants' respiratory rate detection system is depicted. This view of the detection ring assembly 300 shown in FIG. 3(a) is the side facing the patient's nose and mouth. A detection ring 302 is configured to attach to a mask 102. An opening 306 is formed within detection ring 302. During use, air passing to and from a patient's nose and/or mouth passes through the opening 306.

A sensor support 304 is attached to the detector ring 302 and disposed across the opening 306. A microphone-based sensor 308 is attached to the sensor support 304. The microphone-based sensor is positioned at a location, when operationally positioned on a patient, where the stream of air from the nose and the stream of air from the mouth intersect for an average person. In one embodiment, the microphone-based sensor 308 has a pickup 310.

Turning to FIG. 3(b), one embodiment of a microphone based pickup 320 for use in the embodiment of FIG. 3(a) is depicted. A noise maker 322 is attached to a microphone 324 by supports 326 and 328. The support provides space between noise maker 322 and microphone 324 to allow air to flow through noise maker 322. In one embodiment, the noise maker 322 is releasably attached to microphone 324 to allow the noise maker 322 to be replaced for each patient.

The microphone 324 has a pickup 330. The noise maker 322 is attached to the side of the microphone 324 containing the pickup 330. In one embodiment, the noise maker 322 includes a number of thin flexible sheets arranged parallel to each other. Noise maker 322 transforms the movement of exhaled air into noise that can be detected by the microphone pickup 330. As air flows over the sheets, the air causes the sheets to vibrate and rustle. The noise created by this motion is detected by the microphone pickup 330.

The sides of the microphone 324 are sloped away from the noise maker 322. This shape directs the flow of air during inhalation to flow substantially around the noise maker 322, thereby increasing the acoustic signature between an inhalation and an exhalation. The microphone pickup 330 converts the noise into an electrical signal that is fed to a processor. In one embodiment, a breath (i.e., a exhalation/inhalation or inhalation/exhalation pair) is detected by use of a microphone which produces a frequency feed into a non-inverting amplifier circuit which is then counted in a 15 second loop. Once the 15 seconds has expired, the processor calculates the respiratory rate by multiplying the total count by 4.

In one embodiment, the microphone based pickup 320 does not include a noise maker 322. The microphone is configured to directly detect the movement of air from each breath.

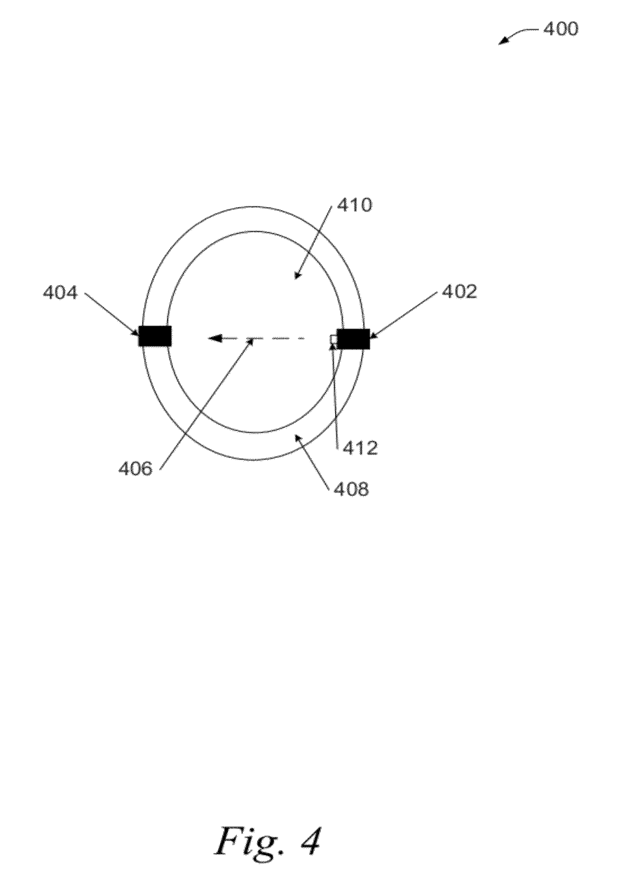

Referring to FIG. 4, one embodiment of a respiratory rate detection ring assembly 400 using an optical flow meter for use in Applicants' respiratory rate detection system is depicted. A detection ring 408 is configured to attach to a mask 102. An opening 410 is formed within detection ring 408. During use, air passing to and from a patient's nose and/or mouth passes through the opening 410.

An optical flow sensor includes a light source 402 and a detector 404 attached to the detection ring 408. The light source 402 and detector 404 are aligned such that the light beam emitted by light source 402 travels across opening 410 and strikes detector 404 as indicated by arrow 406. The light beam emitted by light source 402 produces a continuous light beam that travels perpendicular to the air flowing through the opening 410 during inhalation and exhalation events. As the air flows through opening 410 the detector 404 picks up minute changes in the light beam caused by the flow of air. In one embodiment, the optical flow sensor uses a laser to track the speed of particles in the air flow to determine the speed of the air flow. In one embodiment, the optical flow sensor uses an optical scintillation technique to measure the turbulence found in the air flow to determine the speed of the air flow.

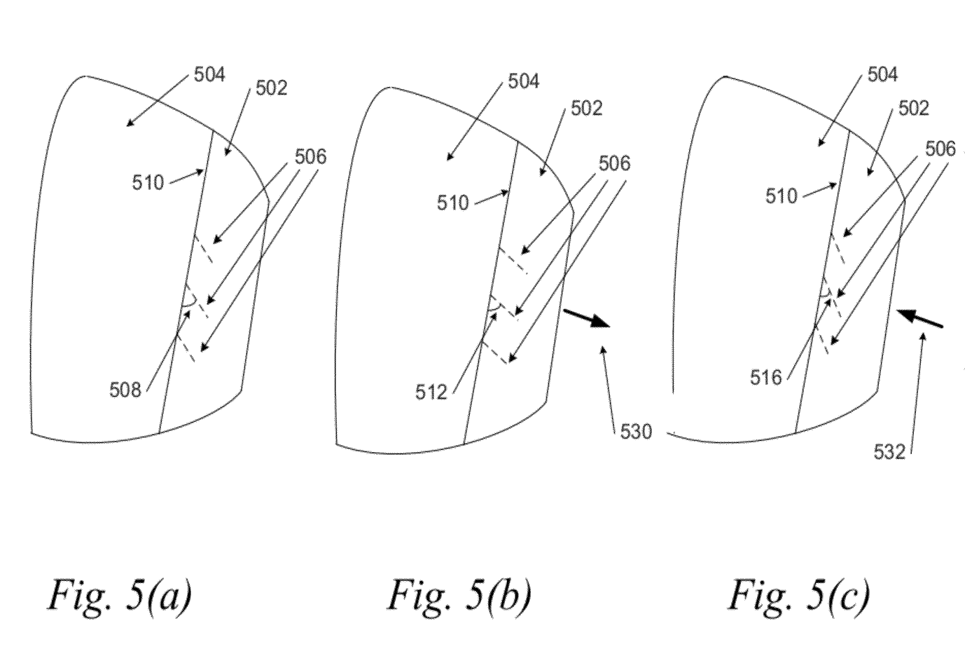

Referring to FIG. 5(a), one embodiment of a respiratory rate detection ring assembly 500 using a vane-based detector for use in Applicants' respiratory rate detection system is depicted. A detection ring 502 is configured to attach to a mask 504. An opening (not shown in this view) is formed within detection ring 502 and is aligned with a matching opening (also not shown in this view) in mask 504. During use, air passing to and from a patient's nose and/or mouth passes through these openings. A support bar 510 is attached to the detection ring 502. The support bar is disposed across the opening of the detection ring 510 and is positioned such that the vanes 506 are in the direct path of air flowing from the nose and mouth of an average person. When at rest, the vanes 506 are positioned at an angle 508 relative to support bar 510. In one embodiment, the angle 508 is about 45 degrees. In one embodiment, the vanes 506 are thin and rigid plastic sheets.

The vanes 506 are attached to the support bar 510 by a mechanism that is capable of detecting the angle 508 of the vanes 506 relative to the support bar 506. In one embodiment, the mechanism includes a piezoelectric material to detect the angle 508. In one embodiment, the mechanism includes electrical contacts that engage when the vanes are at one or more specific angles.

Turning to FIG. 5(b), the embodiment of FIG. 5(a) is shown during an exhalation event. The flow of air is indicated by arrow 530. During an exhalation event, the vanes 506 extend relative to support bar 510, resulting in an angle 512. Angle 512, which occurs as a result of the air flow of an exhalation event, is greater than angle 508 of FIG. 5(a), which occurs when there is no air flow due to inhalation or exhalation. At angle 512, the mechanism attached to support bar 510 detects the change in angle and sends a signal to the processor.

Turning to FIG. 5(c), the embodiment of FIG. 5(a) is shown during an inhalation event. The flow of air is indicated by arrow 532. During an exhalation event, the vanes 506 extend relative to support bar 510, resulting in an angle 516. Angle 516, which occurs as a result of the air flow of an exhalation event, is greater than angle 508 of FIG. 5(a), which occurs when there is no air flow due to inhalation or exhalation. At angle 516, the mechanism attached to support bar 510 detects the change in angle and sends a signal to the processor.

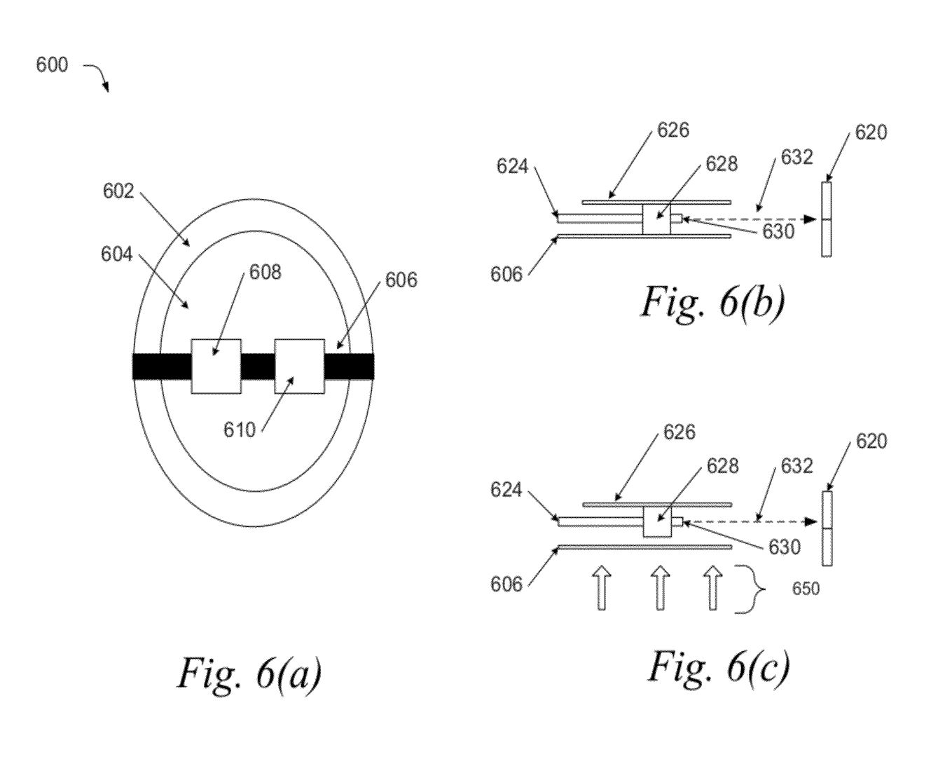

Referring to FIG. 6(a), one embodiment of a respiratory rate detection ring assembly 600 using a contact panel-based detector for use in Applicants' respiratory rate detection system is depicted. A detection ring 602 is configured to attach to a mask 102. An opening 604 is formed within detection ring 602 and is aligned with a matching opening in mask 102. During use, air passing to and from a patient's nose and/or mouth passes through these openings. A support bar 606 is attached to the detection ring 602. The support bar 606 is disposed across the opening 604 of the detection ring 602.

In one embodiment, two contact plate detectors 608 and 610 are attached to support bar 606. In one embodiment, only one contact plate detector 608 is attached to support bar 606 at the midpoint of the support bar 606.

Turning to FIG. 6(b), a side view of the contact plate detector 608 and 610 of FIG. 6(a) is shown. A contact arm 624 is attached to support bar 606 (not shown in current view). A panel mount 628 is attached to contact arm 624. A contact panel 626 is attached to panel mount 628. In one embodiment, the panel mount 628 is in electrical connection with support bar 606.

Turning to FIG. 6(c), the air flow 650 during an exhalation creates a high pressure area under the contact panel 626, causing the contact panel 626 to pull away from the support bar 606. As the contact panel 626 moves in this manner, the panel mount 628 lifts away from the support bar 606 and the electrical contact between the panel mount 628 and the support bar 606 is broken. This causes a signal to be sent to the processor.

Turning back again to FIG. 6(b), in another embodiment, the panel mount 628 includes a light source 630 that projects a beam 632. The beam 623 strikes a detector 620. Turning to FIG. 6(c), the air flow 650 during an exhalation creates a high pressure area under the contact panel 626, causing the contact panel 626 to pull away from the support bar 606. As the contact panel 626 moves in this manner, the panel mount 628 lifts away from the support bar 606 causing the beam 632 to strike a different portion of the detector 620. This causes a signal to be sent to the processor.

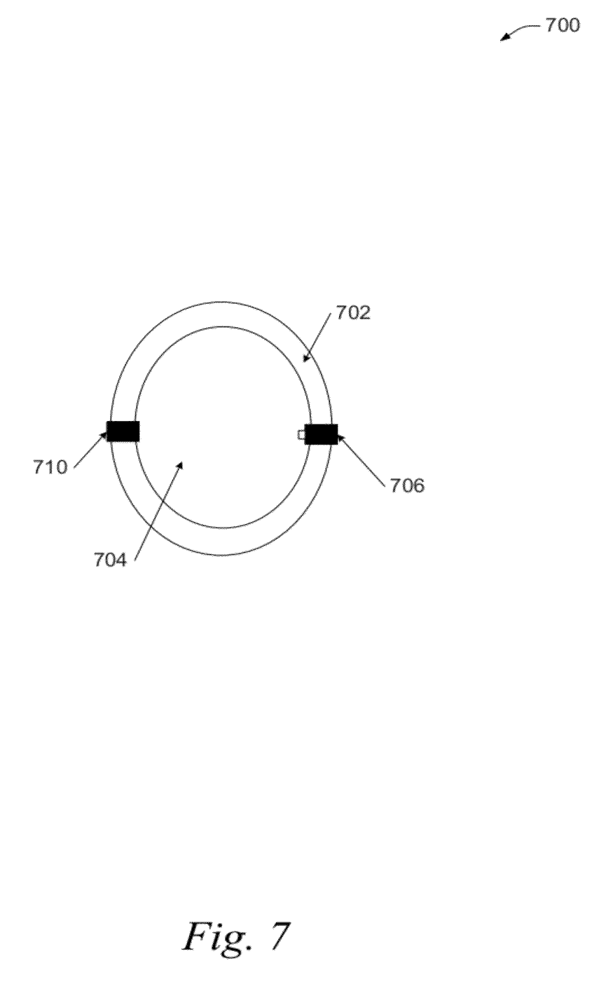

Referring to FIG. 7, one embodiment of a respiratory rate detection ring assembly 700 using a CO₂ detector for use in Applicants' respiratory rate detection system is depicted. A detection ring 702 is configured to attach to a mask 102. An opening 704 is formed within detection ring 702 and is aligned with a matching opening in mask 102. During use, air passing to and from a patient's nose and/or mouth passes through these openings. A CO₂ detector 706 is mounted on the detection ring 702.

In one embodiment, the CO₂ detector 706 is an optical detector using nondispersive infrared technology. In that embodiment, the CO₂ detector 706 requires a second component 710 positioned on the opposite side of the detection ring 702 and in optical alignment with the CO₂ detector 706. In another embodiment, the CO₂ detector 706 is a chemical detector using a thin organic or non-organic film. In this embodiment, the CO₂ detector can detect levels of CO₂ without a second component 710.

The CO₂ detector 706 continuously monitors the level of CO₂ in the opening 704. During exhalation, the level of CO₂ in the air passing through the opening 704 increases. During inhalation, the level of CO₂ in the air passing through the opening 704 decreases. The CO₂ detector provides data containing the level of CO₂ as a function of time to the processor, which then processes the data to determine the respiratory rate.

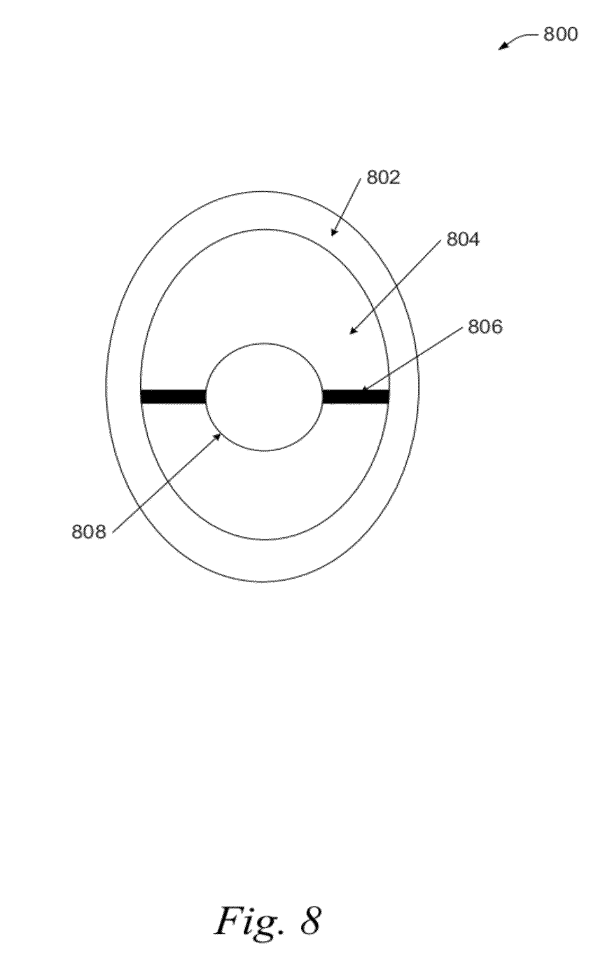

Referring to FIG. 8, one embodiment of a respiratory rate detection ring assembly 800 using a moisture detector for use in Applicants' respiratory rate detection system is depicted. A detection ring 802 is configured to attach to a mask 102. An opening 804 is formed within detection ring 802 and is aligned with a matching opening in mask 102. During use, air passing to and from a patient's nose and/or mouth passes through these openings. A support bar 806 is mounted on the detection ring 802 and across the opening 804. A moisture detector 808 is mounted on the support bar 806.

During exhalation, moisture in the exhaled breath condenses on the surface of the moisture detector 808. Circuitry in the moisture detector 808 registers the increase in moisture on the surface of the moisture detector 808. During inhalation, the incoming dry air will cause the moisture on the surface of the moisture detector 808 to evaporate. The circuitry in the moisture detector 808 registers the decrease in moisture on the surface of the moisture detector 808. The moisture detector 808 provides the moisture level data to the processor.

In another embodiment, the respiratory rate detection ring assembly 800 uses a temperature sensor for respiratory rate detection. A temperature sensor 808 is mounted on the support bar 806. During exhalation, the exhaled breath heats the surface of the temperature sensor 808. Circuitry in the temperature sensor 808 registers the increase in temperature on the surface of the temperature sensor 808. During inhalation, the incoming cool air will cause the temperature on the surface of the temperature sensor 808 to decrease. The circuitry in the temperature sensor 808 registers the decrease in temperature on the surface of the temperature sensor 808. The temperature sensor 808 provides the temperature level data to the processor.

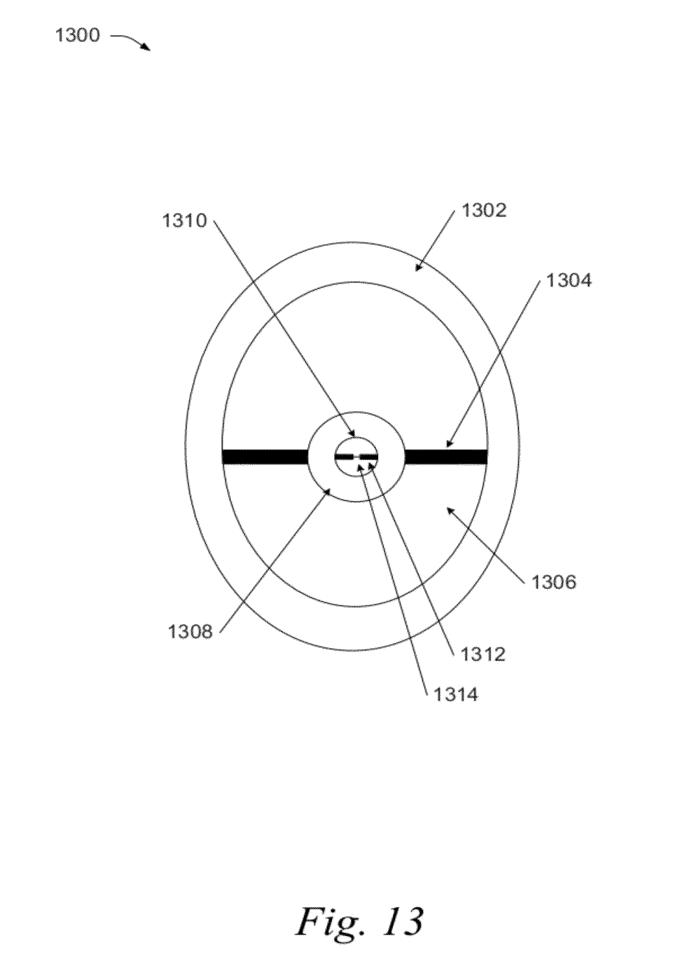

Referring to FIG. 13, one embodiment of a respiratory rate detection ring assembly 1302 using a thermal anemometer-based sensor for use in Applicants' respiratory rate detection system is depicted. A detection ring 1302 is configured to attach to a mask 102. An opening 1306 is formed within detection ring 1302 and is aligned with a matching opening in mask 102. During use, air passing to and from a patient's nose and/or mouth passes through these openings. A support bar 1304 is mounted on the detection ring 1302 and across the opening 1306. A thermal anemometer-based sensor 1308 is mounted on the support bar 1304. In one embodiment, the thermal anemometer-based sensor 1308 includes a hole 1310. A mounting bar 1312 is disposed within the hole 1310. The mounting bar 1312 includes a gap approximately midway along its length. A conductive element 1314 is disposed across the gap in the mounting bar 1312.

The sensor 1308 includes a means for generating a flow of electricity across the conductive element 1314. In one embodiment, the sensor 1308 is a constant-current anemometer (CCA) wherein a constant current is maintained across conductive element 1314. In one embodiment, the sensor 1308 is a constant-temperature anemometer (CTA) wherein the current is adjusted to maintain the conductive element 1314 at a constant temperature. In one embodiment, the sensor 1308 is a constant-voltage anemometer (CVA) wherein a constant voltage is maintained across conductive element 1314. In each type of anemometer (i.e., CCA, CTA, and CVA), the conductive element is heated to a temperature above the ambient temperature. The flow of air over the conductive element 1314 changes the temperature and thus the resistance of the conductive element 1314. This change in resistance, measured using different methods depending on the type of anemometer, is used to detect individual breaths.

In different embodiments, the conductive element 1314 is a thin conductive wire made from, without limitation, tungsten or platinum. In one embodiment, the wire is about 4-10 μm in diameter and about 1 mm in length. In other embodiments, the conductive element 1314 is a conductive film, such as without limitation a platinum film, disposed on a conductive substrate.

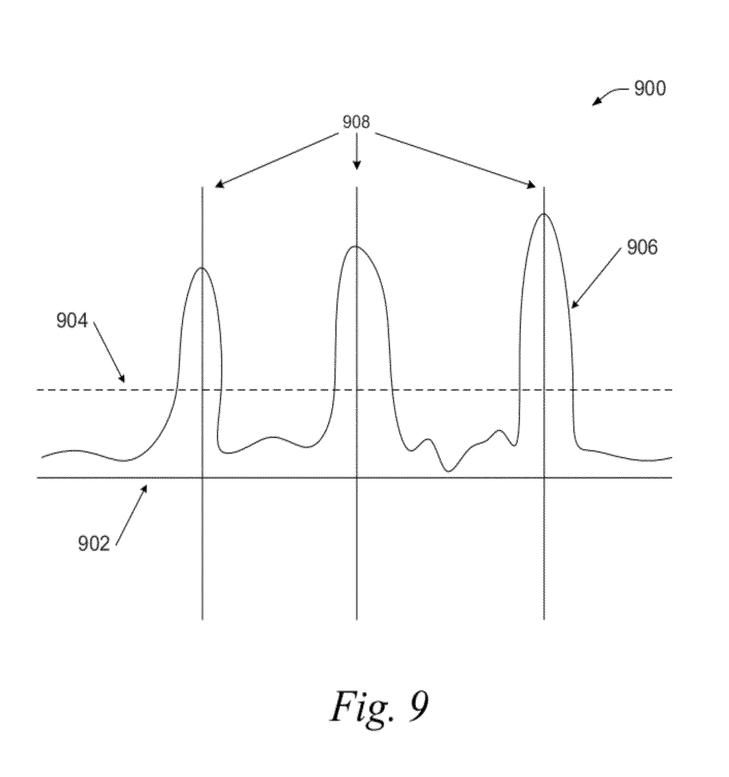

Referring to FIG. 9, a sample graph 900 representing data produced from one of the respiratory sensors described in FIGS. 1-8, 11, and 13. The baseline 902 represents no signal. The trace 906 represents data from the respiratory sensor. Threshold 904 represents the threshold above which an inhalation and/or exhalation will be registered. Depending on the particular embodiment, a dominate peak will result from an exhalation only. For example, the sensor in FIG. 3(b) will produce a much larger peak for exhalations than for inhalations. As such, each exhalation will produce a dominate peak (i.e., much larger than those created by an inhalation), which will be interpreted by the control unit or the processor as a single breath. In other embodiments, an equivalent peak will be generated with both inhalation and exhalation events. In which case, a pair of peaks will be interpreted by the control unit or processor as a single breath. Referring again to FIG. 9, the peaks, marked by vertical lines 908, each represent the occurrence of a single breath.

In one embodiment, a control unit in communication with Applicants' respiratory rate detection unit comprises a processor and a computer readable medium comprising computer readable program code disposed therein to calculate a respiration rate. In one embodiment, the processor causes a timer to initiate. In one embodiment, the timer is set at a predetermined time interval. The processor receives respiration event data from the sensor. In different embodiments, the sensor is of the type depicted in FIG. 1-8, 11, or 13. In different embodiments, the processor filters the respiration event data to remove background noise and to identify an individual respiration cycle. Depending on the type of sensor used, an individual respiration cycle will be the detection of a single inhalation or a single exhalation, or the detection of a inhalation/exhalation pair or a exhalation/inhalation pair. For each individual respiration cycle, the processor increments a breath count variable.

After the timer indicates that the predetermined time interval has elapsed, the processor calculates a respiration rate by determining the number of predetermined time intervals per minute and then dividing the value of the breath count variable by the number of predetermined time intervals per minute. In one embodiment, the processor then resets the breath count variable to zero, resets and restarts the timer, and repeats the process.

In one embodiment, the predetermined time interval is 15 seconds, making the number of predetermined time intervals per minute 4. As such, the timer triggers the processor to calculate the respiration rate every 15 seconds by dividing the number of individual respiration cycles detected in a 15 second period by 4. In some embodiments, the predetermined time interval varies in different stages. For example, when first applied to a subject (i.e., a first stage), the predetermined time interval may be relatively long (for example without limitation, 15 or more seconds) to get an initial accurate respiration rate. After an initial respiration rate is determined (i.e., the second stage), the predetermined time interval may be shortened (for example without limitation, less than 15 seconds) to obtain a more real-time respiration rate.

In one embodiment, in the second stage, the respiration rate is recalculated with each individual respiration cycle detected using a set of the most recent consecutive individual respiration cycles. In various embodiments, the set of the most recent consecutive individual respiration cycles is between 5 and 15 individual respiration cycles. For example, for each individual respiration cycle detected, the processor will recalculate the respiration rate based on the time it took to detect the last 10 individual respiration cycles by dividing 10 by the elapsed time in seconds (as measured by the timer) for the 10 individual respiration cycles to occur and multiplying the result by 60 to adjust to breaths per minute. This gives an updated respiration rate with every breath.

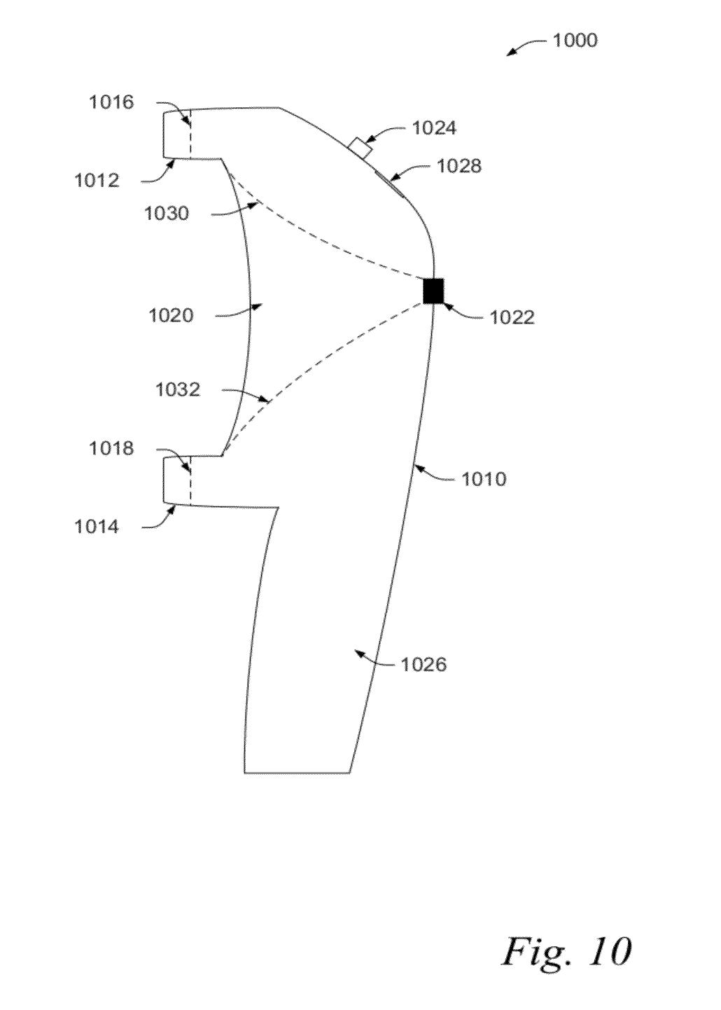

Referring to FIG. 10, one embodiment of a handheld respiratory rate detection system 1000 is depicted. The handheld respiratory rate detection system 1000 includes a body 1010. A upper contact point 1012 and a lower contact point 1014 extend from the body 1010. In one embodiment, the upper and lower contact points 1012 and 1014 have a concave surface that extends into the body 1010. The apex of the surface is represented by broken lines 1016 and 1018. In one embodiment, the surface on the upper contact point 1012 is configured to contact the nose of a patient and the surface on the lower contact point 1014 is configured to contact the chin of a patient. This design permits the respiratory rate detection system 1000 to be properly positioned over the patient's nose and mouth while minimizing physical contact with the patient.

In one embodiment, a disposable cover is releasably attached over each of the upper and lower contact points 1012 and 1014. After the respiratory rate detection system 1000 is used on a particular patient, the disposable cover, which is the only part of the system 1000 in contact with the patient's face, is removed and discarded to maintain hygienic conditions.

A conical cavity 1020 is formed in the body 1010 and indicated by broken lines 1030 and 1032. A sensor 1022 is positioned at the vertex of the conical cavity 1020. In one embodiment, the body 1010 contains an embedded control unit, including a processor and a digital to analog convertor (DAC). The DAC converts the analog signals from the sensor to digital signals to be processed by the processor. In different embodiments, the body contains electronics to format and transmit the measured respiratory rate information to an external display device, such as a digital display or vital stand apparatus. In one embodiment, the information is transmitted through a wired connection. In one embodiment, the information is transmitted through a wireless connection that conforms with a wireless standard, such as without limitation Bluetooth (IEEE 802.15.1 and later implementations), Wi-Fi (IEEE 802.11), irDA, implementations of IEEE 802.15.4 (ex., ZigBee), and Z-Wave. In one embodiment, the information is transmitted by acoustic means, such as by modulating data on an acoustic carrier broadcast. In certain embodiments, the acoustic carrier is in the audible range (between about 20 Hz and about 20 kHz). In certain embodiments, the acoustic carrier is in the inaudible range (below about 20 Hz and above about 20 kHz).

When positioned on a patient and as the patient breathes, air and noise from each exhalation is directed down the conical cavity 1020 and toward the sensor 1022. The shape of the conical cavity 1020 acts to amplify the motion of the air and sound created by each exhalation. The sensor 1022 detects the movement of air or sound, converts it into an electronic signal, and sends the signal to the control unit. In one embodiment, an indicator 1024 provides a visual indication of the state of the respiratory rate detection system 1000.

In one embodiment, openings (not shown) are formed in the body near the apex of the conical cavity 1020 in close proximity to the sensor 1022. The openings allow air to pass through the body 1010 when a patient exhales, preventing a high pressure region to develop at the apex of the conical cavity that may decrease the ability of the sensor 1022 to detect the patient's breath.

To detect respiratory rate, the respiratory rate detection system 1000 is placed over the patient's nose and mouth with the upper contact point 1012 contacting the patient's nose and the lower contact point 1014 contacting the patient's chin. In one embodiment, once activated (which may comprise being switched on from an off state or being woken from a low power state) the indicator 1024 signals that the system 1000 is ready to measure a respiratory rate by appearing blue.

The system 1000 is then placed over the nose and mouth of a patient. As the system 1000 detects a breath, the indicator 1024 changes to yellow. When enough information is collected by the sensor 1022 to determine a respiratory rate for the patient, the indicator 1024 changes to green. In one embodiment, the time necessary to determine respiratory rate once the first breath is detected is 15 seconds. The measured respiratory rate is then displayed on the display 1028. In one embodiment, the respiratory rate, measured in this fashion, is included in the routine procedure for taking vital signs (i.e., temperature, blood pressure, pulse oximetry, etc.) in a health care setting.

In one embodiment, the sensor 1022 is the sensor depicted in FIG. 3(b). In other embodiments, the sensor 1022 is the sensor depicted in FIGS. 4, 5(a)-5(c), 6(a)-6(c), 7, 8, and 13. In one embodiment, one part of the body is elongated into a handle 1026. In one embodiment, the handle 1026 enables the respiratory rate detection system 1000 to be held in place by the patient or a medial practitioner during the measurement period.

In one embodiment, the system 1000 is coupled with a carbon dioxide detector. In one embodiment, the system 1000 is coupled with an oxygen detector. In one embodiment, the system 1000 is coupled with an alcohol detector.

In various embodiments, the sensors depicted in FIGS. 3(a)-3(b), 4, 5(a)-5(c), 6(a)-6(c), 7, 8, and 13 are coupled with a carbon dioxide detector, an oxygen detector, an alcohol detector, or a combination thereof.

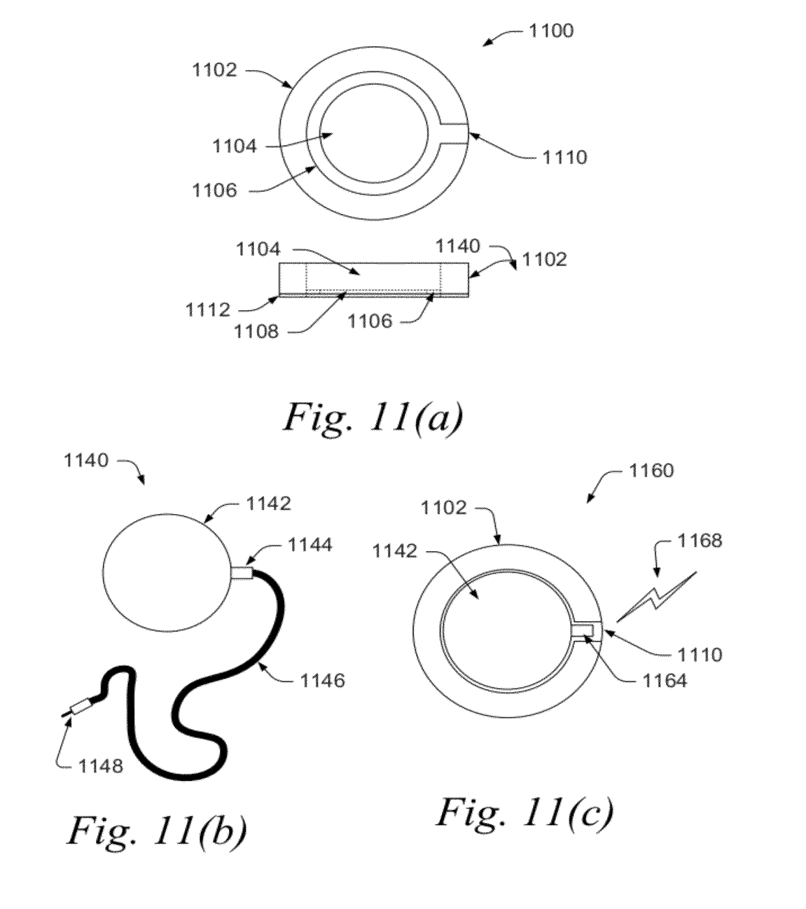

Referring to FIG. 11(a), a disposable adhesive adapter 1100 is depicted. The adapter 1100 is designed to receive a respiratory rate sensor. The adaptor 1100 comprises an outer ring 1102 with a gap 1110. An adhesive strip 1112 is attached to the bottom of the outer ring 1102. A hole 1104 is formed through the outer ring 1102 and adhesive strip 1112. A lip 1106 is disposed within the hole 1104. In one embodiment, a thin membrane 1108 is disposed within and across the hole 1104.

Referring to FIG. 11(b), a wired respiratory rate sensor 1140 for use with the disposable adhesive adapter 1100 of FIG. 11(a) is depicted. The sensor 1140 includes a sensor body 1142. The sensor body 1142 detects each breath of a patient. In one embodiment, the sensor body 1142 includes a high frequency diaphragm, such as those used in a traditional stethoscope chestpiece. In one embodiment, the sensor 1142 includes a microphone. In one embodiment, the sensor 1142, similar to the chestpiece of an electronic stethoscope, includes a piezoelectric crystal or an electromagnetic diaphragm.

A stem 1144 is attached to the sensor 1142. The sensor 1142 and stem 1144 are designed to fit within the hole 1104 and gap 1110 of the adapter 1100 and rest against the lip 1106. The sensor is secured to the adapter by mechanical means.

A wire 1146 is attached to the stem 1144. A connector 1148 is attached to the wire 1146. The connector 1148 is configured to connect to a display device or vital stand apparatus for displaying the measured respiratory rate.

Referring to FIG. 11(c), a wireless respiratory rate sensor 1160 attached to the disposable adhesive adapter 1100 of FIG. 11(a) is depicted. In one embodiment, the sensor body 1142 has an antenna 1164. The sensor body 1142 is disposed in the outer ring 1102 and the antenna 1164 is disposed in the gap 1110. The respiratory rate information acquired by the sensor body 1142 is communicated wirelessly by signal 1168 to a control unit, processor, display unit, or vital stand apparatus.

In one embodiment, the wireless respiratory rate sensor 1160 is disposed over a patient's windpipe. In one embodiment, the wireless respiratory rate sensor 1160 is disposed over one of the patient's lungs. The adhesive strip 1112 secures the wireless respiratory rate sensor 1160 to the patient. When attached via the adhesive strip, a chamber is created between the patient's body and the thin membrane 1108. Sound generated by the breathing action of the patient passes through the chamber and is propagated to the sensor body 1142 by the thin membrane 1108. In one embodiment, the thin membrane 1108 enhances the sound so it can be better detected by the sensor body 1142.

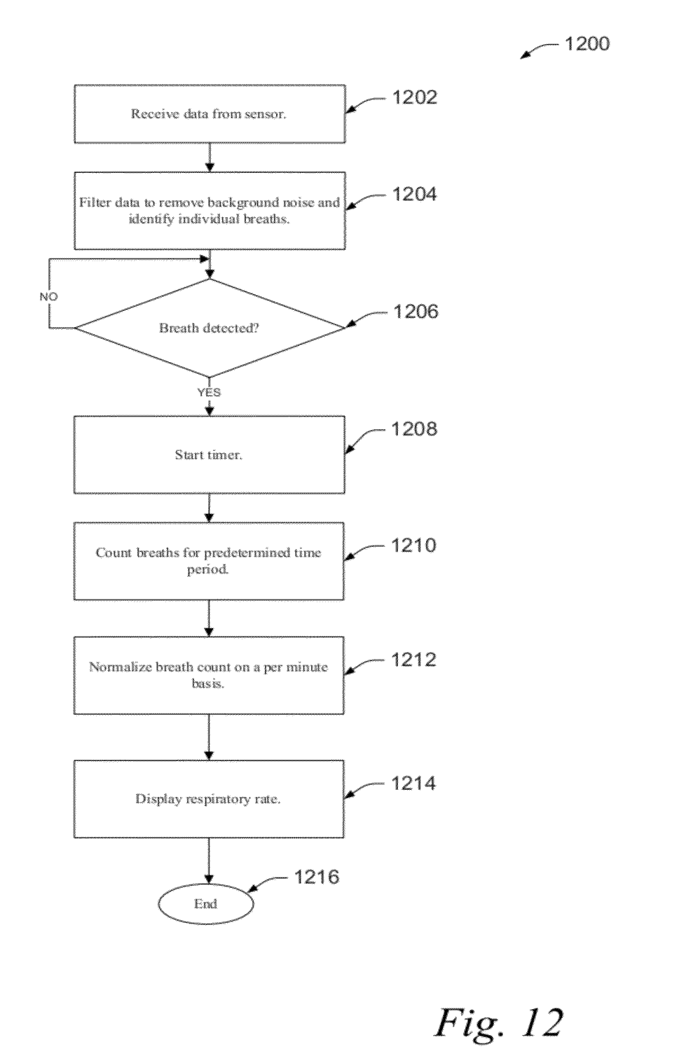

Referring to FIG. 12, a flowchart 1200 showing the steps of determining the current respiratory rate of a patient by using Applicants' respiratory rate detection system is depicted. Data from a sensor is received at step 1202. The data is filtered at step 1204 to remove background noise and isolate the signals indicative of a breath. The method decides if a breath has been detected at step 1206. If the method determines that a breath has not been detected, the method loops back to step 1206.

If the method determines that a breath has been detected, the method transitions to step 1208. A timer is triggered at step 1208. The method counts breaths for a predetermined time period at step 1210. In different embodiments, the time period is at or between 10 seconds and one minute. In one embodiment, the time period is automatically determined based on the signal to noise ratio from the sensor; the time period is increased for lower signal to noise ratios and the time period is decreased for higher signal to noise ratios.

The respiratory rate per minute is determined by normalizing the breath count on a per minute basis at step 1212. The respiratory rate is displayed at step 1214. The method ends at step 1216.

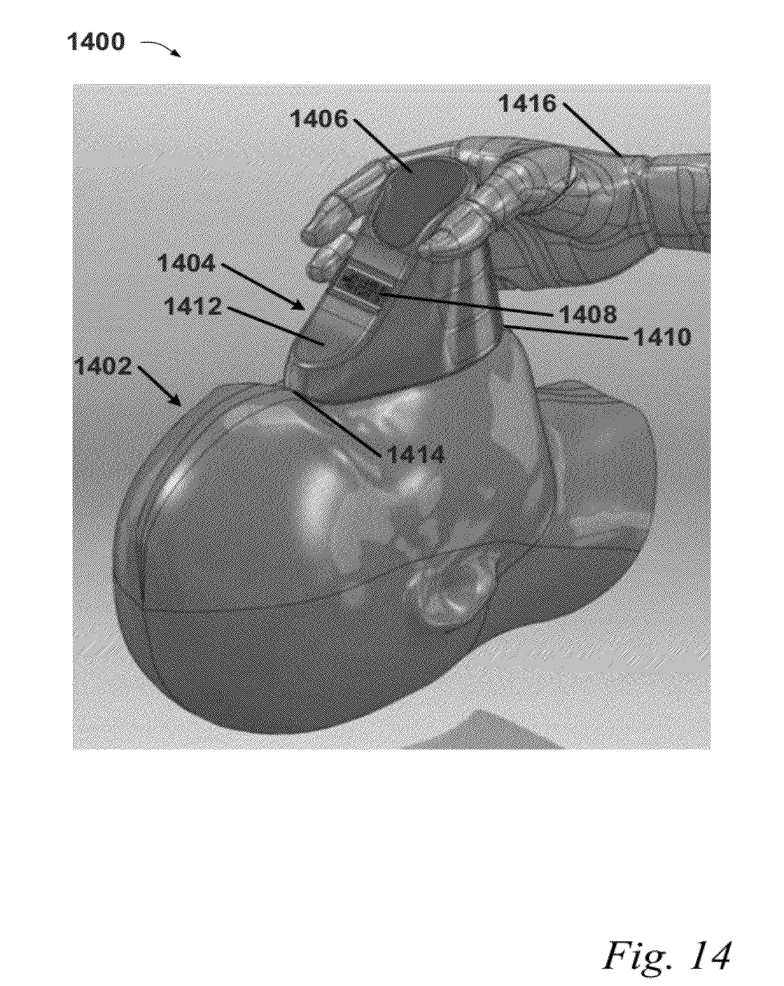

Referring to FIG. 14, a three dimensional view 1400 of one embodiment of Applicants' respiratory rate detection system 1404 is depicted. A device housing 1412 is configured to be disposed over the mouth and nose of a subject 1402. In one embodiment, the device housing is tubular with a channel formed therein to direct the flow of air from the subject's nose and mouth.

The device housing 1412 houses a sensor capable of detecting the airflow generated as the subject 1402 breaths. In various embodiments, the sensor may be any sensor described herein, including a sensor comprising a microphone (as described in FIGS. 3(a)-3(b)), an optical detector (as described in FIG. 4), a system of vanes (as described in FIGS. 5(a)-5(c)), a contact panel (as described in FIGS. 6(a)-6(c)), a carbon dioxide detector (as described in FIG. 7), a moisture or temperature sensor (as described in FIG. 8), a pulse detector (as described in FIGS. 11(a)-11(c), a thermal anemometer (as described in FIG. 13), or a combination thereof.

In one embodiment, the status of the respiratory rate detection system is displayed on a display panel 1408. In various embodiments, the information on the display will include, without limitation, the instant respiratory rate for the subject 1402, the average respiratory rate over a time period for the subject 1402, any alerts or alarms, the battery strength, the strength of the wireless communication signal, a breath indicator (to instantaneously indicate the detection of a breath), or a combination thereof. In different embodiments, the screen of the display panel 1408 is an LCD, LED, or OLED display. In one embodiment, the display panel 1408 is touch sensitive to allow the user to operate the respiratory rate detection system 1404 by touching the display panel 1408.

In certain embodiments, the respiratory rate detection system 1404 is countouredly shaped to conform to typical facial features. In one embodiment, the respiratory rate detection system 1404 is supported on the face at the saddle of the nose 1414 (where the top portion of the nose meets the forehead) and the chin 1410. In one embodiment, when the portion of the respiratory rate detection system 1404 is in contact with the saddle of the nose 1414, the portion that contacts the chin 1410 is configured to contact slightly below the lower lip on a subject 1402 having a small head and is configured to contact the lower part of the chin on a subject 1402 having a large head.

In one embodiment, the respiratory rate detection system 1404 is manually held in place by a hand 1416. In another embodiment, the respiratory rate detection system 1404 is secured to the face with a strap (not shown). In one embodiment, respiratory rate detection system 1404 is configured to rest on the face of a subject 1402 without being manually held in place.

A channel 1406 is formed through the respiratory rate detection system 1404. The channel has a distal opening 1508 and a proximal opening 1422. As the subject 1402 breaths, the action of the subject's lungs create a transient pressure event (i.e., a pressure increase (exhalation) or pressure decrease (inhalation)) at the proximal opening 1422. The transient pressure event is received by the proximal opening, which is configured to communicate with the subject's nose and mouth, and results in a flow of air through the channel 1406. Air flows to the distal opening 1422 during an exhalation and from the distal opening 1422 during an inhalation.

In certain embodiments, the distal opening 1508 opens to ambient atmosphere, allowing the free and unobstructed flow of air out of the distal opening 1508. In certain embodiments, the distal opening 1508 is open and is not connected to a tube, confined channel, or other apparatus. In certain embodiments, flow of air in the channel allows the free flow of air to and from the subject's lungs with substantially no resistance. In other embodiments, the distal opening 1508 is in fluid communication with a breathing tube attached to, for example without limitation, an oxygen delivery system.

The sensor is configured to detect a respiration event by monitoring the flow of air through the channel 1406. In certain embodiments, the sensor is disposed within the device housing. In certain embodiments, the sensor is disposed within the channel.

In various embodiments, the respiratory rate detection system 1404, using a wireless communication unit, wirelessly communicates with an external device, such as without limitations, a computer, a vital stand, a remote handheld detector, an internet enabled device, or a combination therein. In one embodiment, the respiration rate is displayed on a display external to the respiration rate detection system 1404, including without limitation, a computer, a vital stand, a remote handheld detector, an internet enabled device, or a combination therein.

In one embodiment, the portion of the respiratory rate detection system 1404 that contacts the face of the subject 1402 is covered with a disposable liner. In one embodiment, the channel 1406 is lined with a disposable liner.

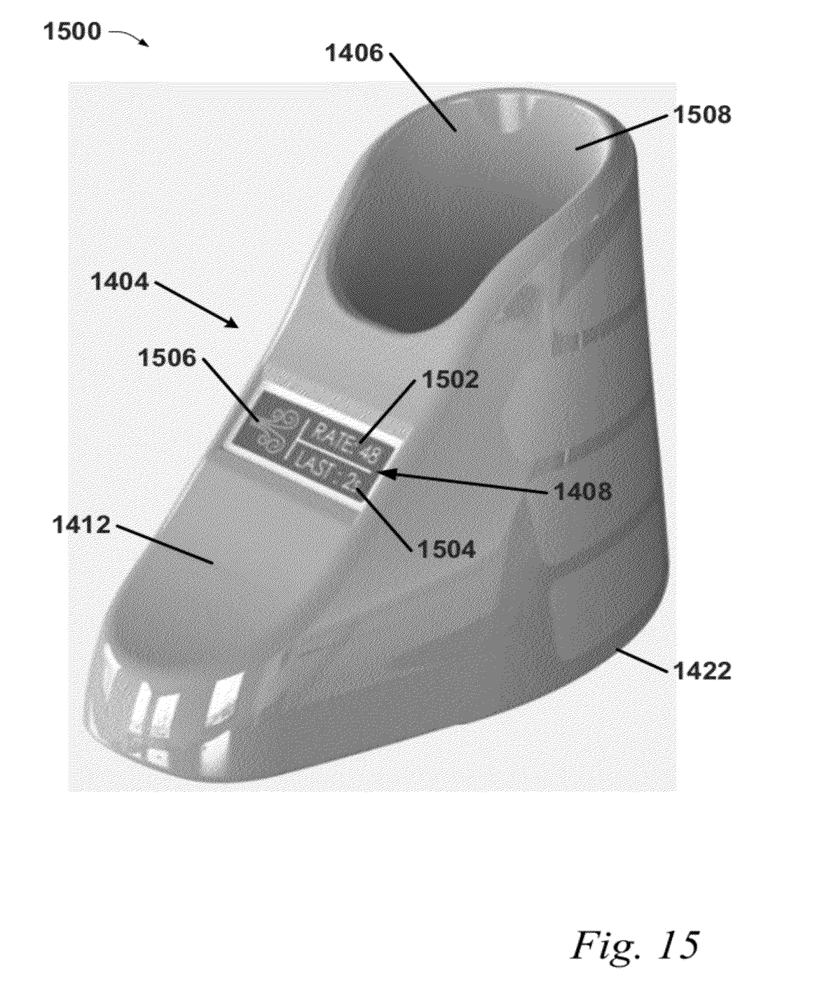

Referring to FIG. 15, an additional three dimensional view 1500 of one embodiment of Applicants' respiratory rate detection system described in FIG. 14 is depicted. In one embodiment, the display panel 1408 is a digital display that displays the average respiration rate 1502, the length of time that has elapsed 1504 since the last breath, and a breath indicator 1506 that indicates the instant and real-time occurrence of a breath. In certain embodiments, a control unit, comprising a processor, a computer readable medium comprising computer readable program code disposed therein, a battery, and other electrical components, is housed in a cavity (not visible) within the device housing 1412. The processor utilizes the computer readable program code to determine the respiration rate of the subject based on the date from the sensor. The battery supplies power to the electrical components of the respiratory rate detection system, including where applicable, the processor, display, battery, and wireless control unit. The processor receives respiration events from the sensor and calculates and displays the respiration rate on the display 1506.

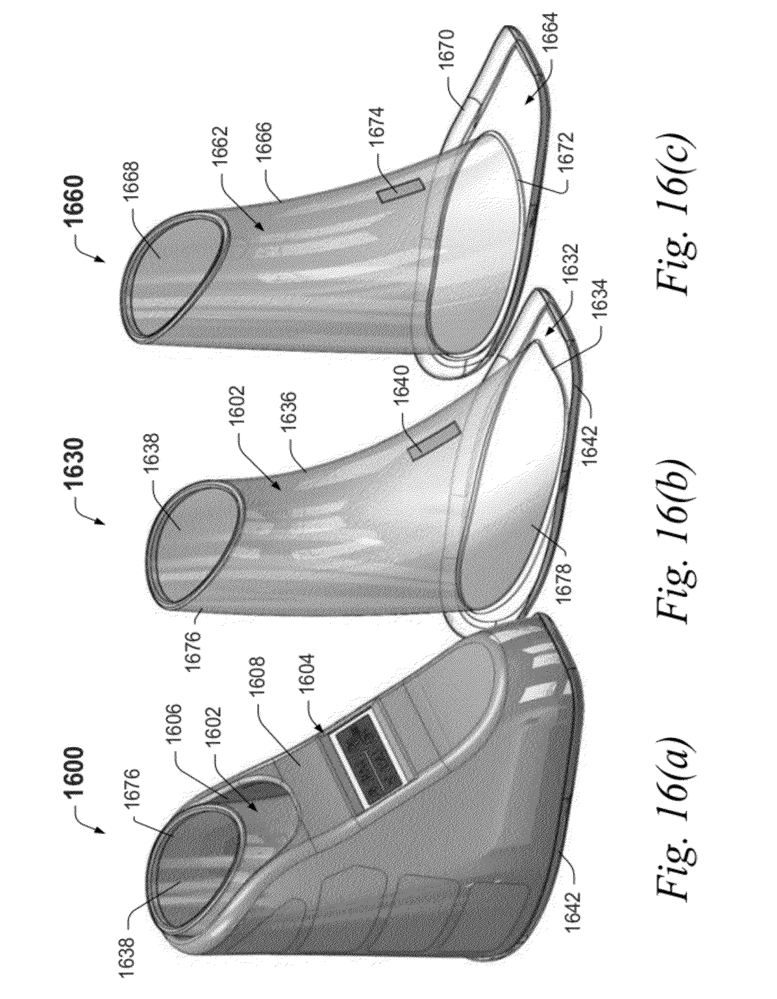

Referring to FIG. 16(a), a three dimensional view 1600 of one embodiment of Applicants' respiratory rate detection system with a sanitary liner 1602 is depicted. The liner 1602 has a distal end 1676 and a proximal end 1678 (shown in FIG. 16(b)). The liner 1602 is disposed within the channel 1606 of the device housing 1608. In one embodiment, the distal end 1676 of liner 1602 extends beyond the distal opening of channel 1606. In one embodiment, the opening in the distal end 1676 of the liner 1602 is selected based on one or more attributes of the subject, such as without limitation, age, weight, and respiratory condition. In one embodiment, the opening in the distal end 1676 of the liner 1602 has a maximum dimension of about 1 inch.

The liner 1602 is configured to conform to and removably, but securely, attach to the device housing 1608 of the respiratory rate detection unit 1604. The liner 1602 is further configured to cover all portions of the respiratory rate detection unit 1604 that come into contact with a subject's face during respiration rate measurement. The liner 1602 is also configured to line channel 1606 of the respiratory rate detection unit 1604.

The liner 1602 is configured to comfortably contact the subject's face during respiration rate measurement. In one embodiment, the liner 1602 is formed from a latex-free and DEHP-free material. In one embodiment, the liner 1602 comprises an acrylic material. In one embodiment, the liner 1602 comprises a hydrogel. In one embodiment, the liner 1602 is configured to act as a barrier to bacteria, viruses, and other pathogens. In one embodiment, the liner 1602 comprises a material that actively neutralizes pathogens. After measurement of a first subject's respiration rate, the liner 1602 is configured to be detached from the respiratory rate detection unit 1604, discarded, and replaced with a new liner before the measurement of a second subject's respiration rate.

Channel 1606 defines an internal volume (the total volume of air within the channel) and the liner 1602 defines an internal volume. In one embodiment, the internal volume of the liner 1602 is selected based on one or more attributes of the subject, such as without limitation, age, weight, and respiratory condition. In certain embodiments, the internal volume of the liner 1602 is greater than about 95% of the internal volume of the channel 1606. In certain embodiments, the internal volume of the liner 1602 is less than about 20% of the internal volume of the channel 1606. In certain embodiments, the internal volume of the liner 1602 is about 20% to about 95% of the internal volume of the channel 1606.

Referring to FIG. 16(b), one embodiment of the liner 1602 from FIG. 16(a), removed from the respiratory rate detection unit 1604, is depicted. The liner has a distal end 1676 and a proximal end 1678. In one embodiment, the liner 1602 comprises a base 1632 attached to a tubular section 1636 at intersection 1634. In one embodiment, the base 1632 is unitarily formed with the tubular section 1636. In another embodiment, the base 1632 and tubular section 1636 are separately formed and attached. In one embodiment, the base 1632 comprises a lip 1642, which curls over the outer base of the respiratory rate detection unit 1604. In one embodiment, the lip 1642 is configured to snap onto the base of the respiratory rate detection unit 1604 to secure the liner 1602 in place during use.

A channel 1638 is formed by the tubular section 1636 through the base 1642 to allow air to flow through the liner 1602. In one embodiment, a sensor port 1640 is formed on the side of the tubular section 1636. The size, shape, and placement of the sensor port is determined by the type and location of the sensor within the respiratory rate detection unit 1604. In one embodiment, as the liner 1602 is inserted into the unit 1604 and secured in place, the sensor protrudes through the sensor port 1640.

In another embodiment, the sensor is non-removably integrated into liner 1602. In this embodiment, there is no sensor port and the liner serves as an uninterrupted barrier along the entire length of the tubular section 1636. In one embodiment, the sensor is in electrical communication with the respiratory rate detection unit 1604 by way of electrical contacts that pass through the liner 1602. In one embodiment, the sensor is in wireless communication with the respiratory rate detection unit 1604 by way of a wireless transmission unit integrated into the detection unit.

Liner 1602 is configured for use with an adult subject. The opening in the base 1632 defined by the intersection 1634 of the base 1632 and the tubular section 1636 is relatively large in area to accommodate the larger facial features of an adult. Referring to FIG. 16(c), one embodiment of a liner 1602 configured for use by a child, to be used in respiratory rate detection unit 1604, is depicted. The liner 1662 shares the same general features of embodiment 1602 depicted in FIG. 16(b), including a base 1664 attached to a tubular section 1666 at intersection 1672, a lip 1670, a channel 1668, and a sensor port 1674. The opening in the base 1664 defined by the intersection 1672 of the base 1664 and the tubular section 1666, however, is smaller in area to accommodate the smaller facial features of a child and to better direct the lower volumetric flow of air over the sensor.

In another embodiment, a sensor is integrated into liner 1662. In this embodiment, there is no sensor port and the liner serves as an uninterrupted barrier along the entire length of the tubular section 1666. In one embodiment, the sensor is in electrical communication with the respiratory rate detection unit 1604 by way of electrical contacts that pass through the liner 1662. In one embodiment, the sensor is in wireless communication with the respiratory rate detection unit 1604 by way of a wireless transmission unit integrated into the detection unit.

Referring to FIG. 17(a), a front, right, top perspective view 1700 of one embodiment of a respiratory rate detection unit 1702 is presented.

Referring to FIG. 17(b), a front, right, top perspective view 1750 of one embodiment of a respiratory rate detection unit 1702 with a removable liner 1754 is presented.

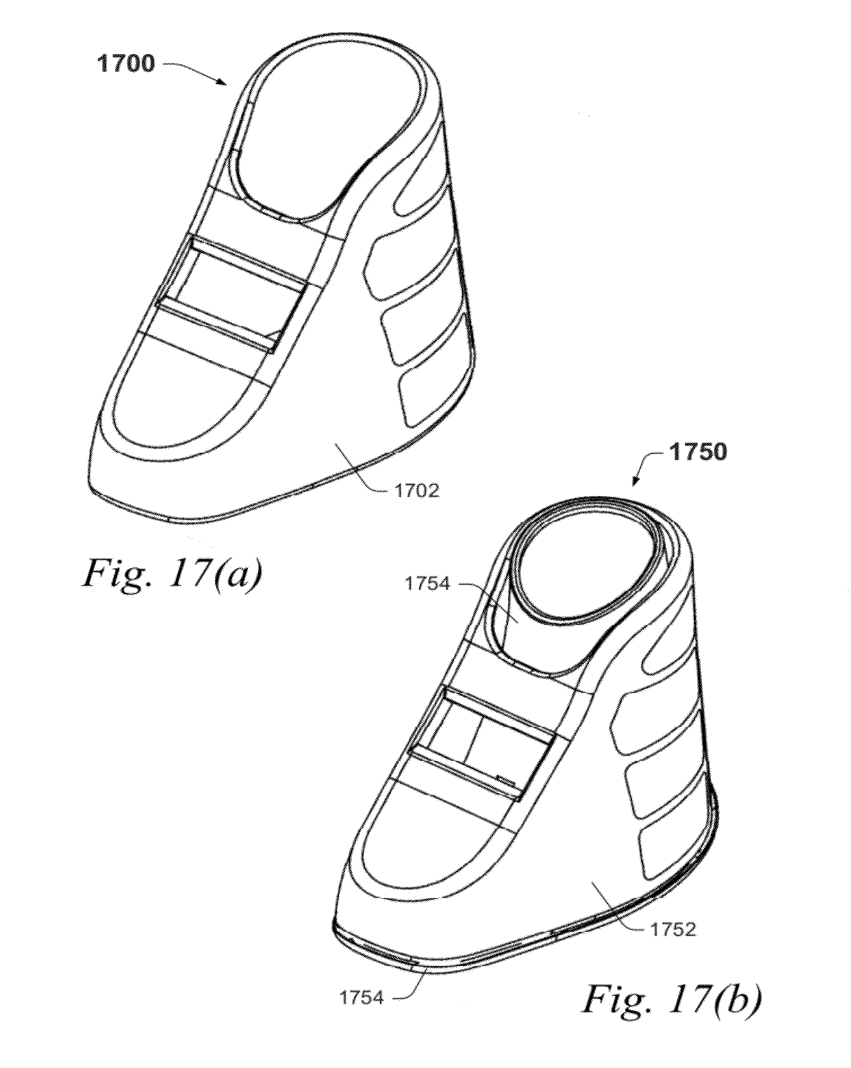

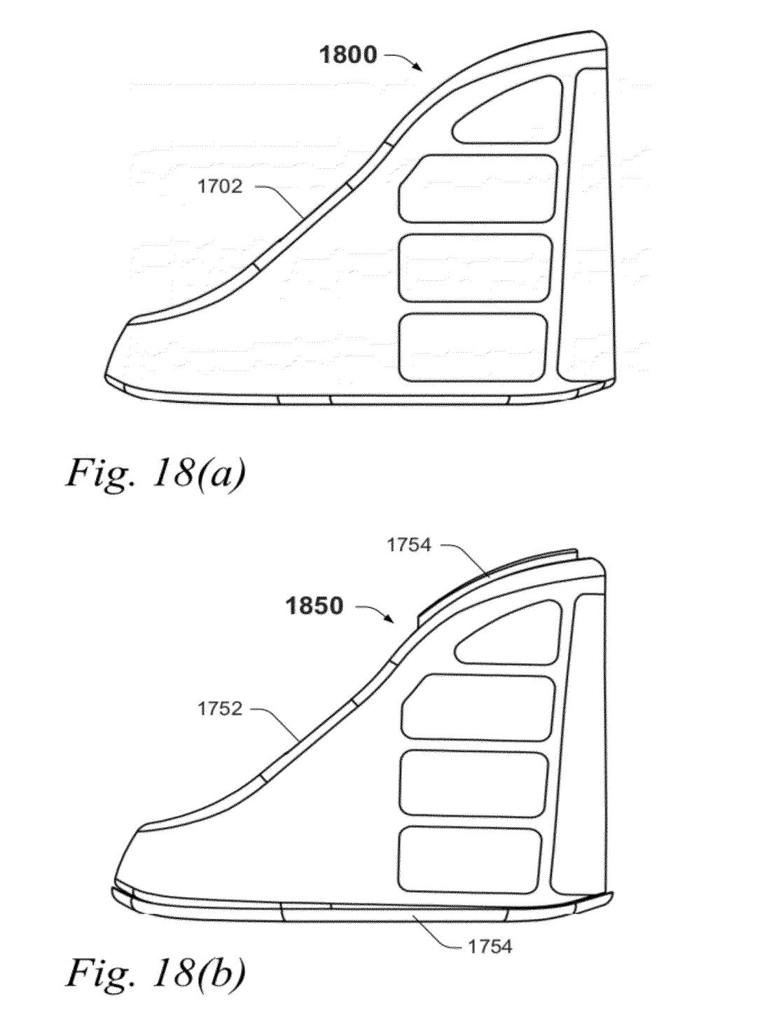

Referring to FIG. 18(a), a right side elevational view 1800 of one embodiment of a respiratory rate detection unit 1702 is presented.

Referring to FIG. 18(b), a right side elevational view 1850 of one embodiment of a respiratory rate detection unit 1752 with a removable liner 1754 is presented.

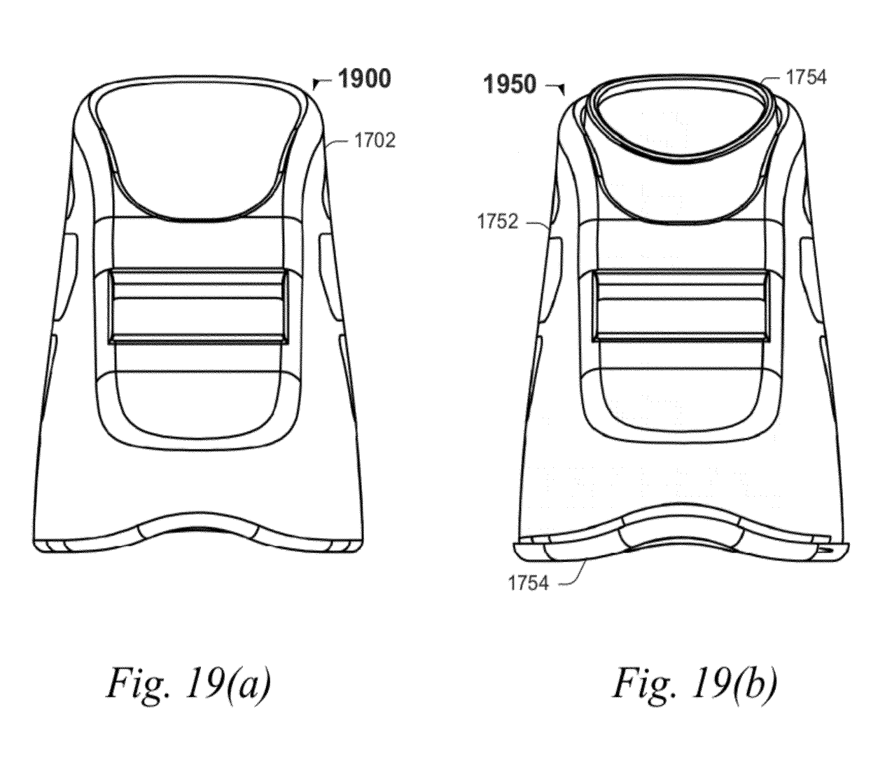

Referring to FIG. 19(a), a front elevational view 1900 of one embodiment of a respiratory rate detection unit 1702 is presented.

Referring to FIG. 19(b), a front elevational view 1950 of one embodiment of a respiratory rate detection unit 1702 with a removable liner 1754 is presented.

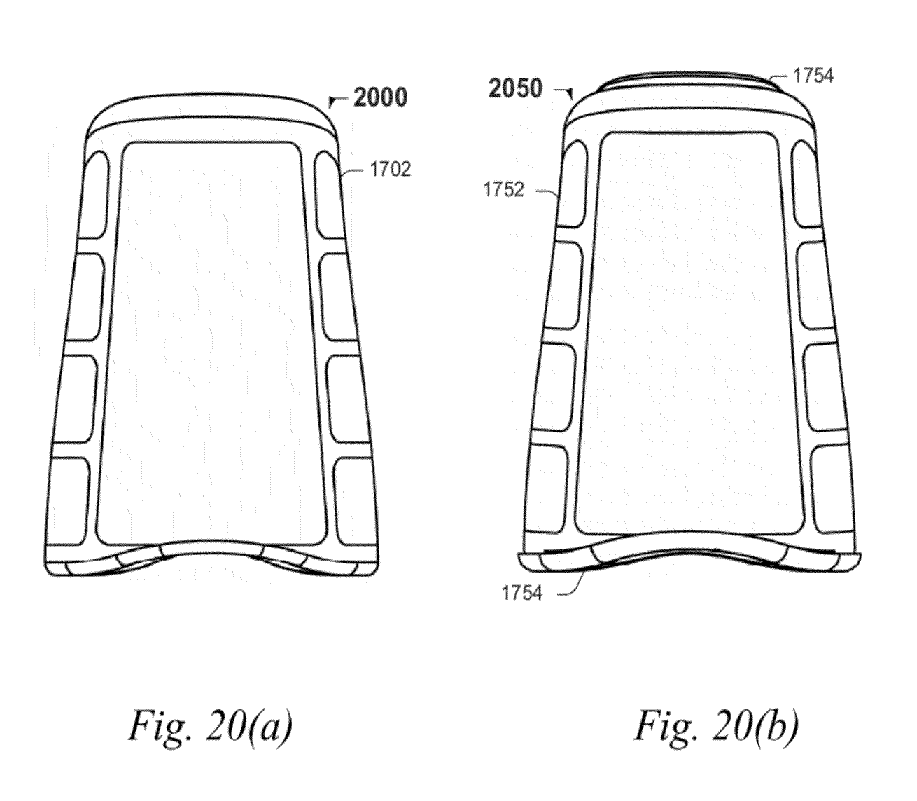

Referring to FIG. 20(a), a rear elevational view 2000 of one embodiment of a respiratory rate detection unit 1702 is presented.

Referring to FIG. 20(b), a rear elevational view 2050 of one embodiment of a respiratory rate detection unit 1702 with a removable liner 1754 is presented.

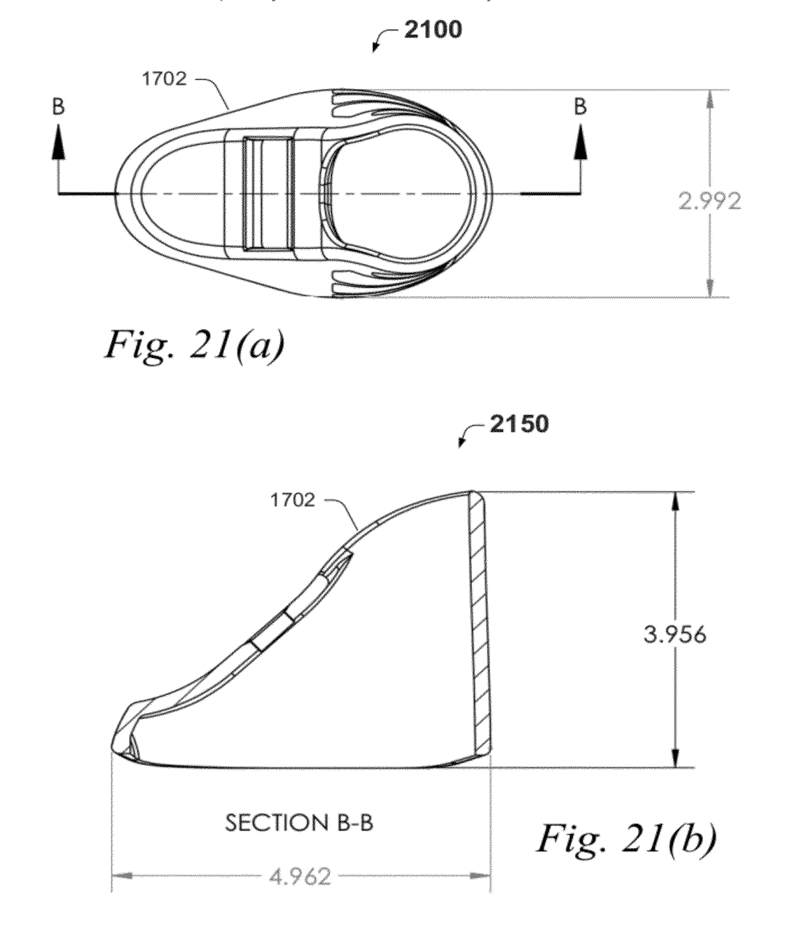

Referring to FIG. 21(a), a top plan view 2100 of one embodiment of a respiratory rate detection unit 1702 is presented. In the embodiment shown, the respiratory rate detection unit 1702 has a width of about 3 inches as indicated on FIG. 21(a).

Referring to FIG. 21(b), a right side cross sectional view 2150 of one embodiment of a respiratory rate detection unit 1702 is presented. The cross section is taken along section line B-B depicted in FIG. 21(a) with the front portion removed so the interior cavity of the respiratory rate detection unit can seen. The sensor and other internal components have been omitted to better illustrate the structure of the device housing. In this embodiment shown, the respiratory rate detection unit 1702 has a height of about 4 inches as indicated on FIG. 21(b).

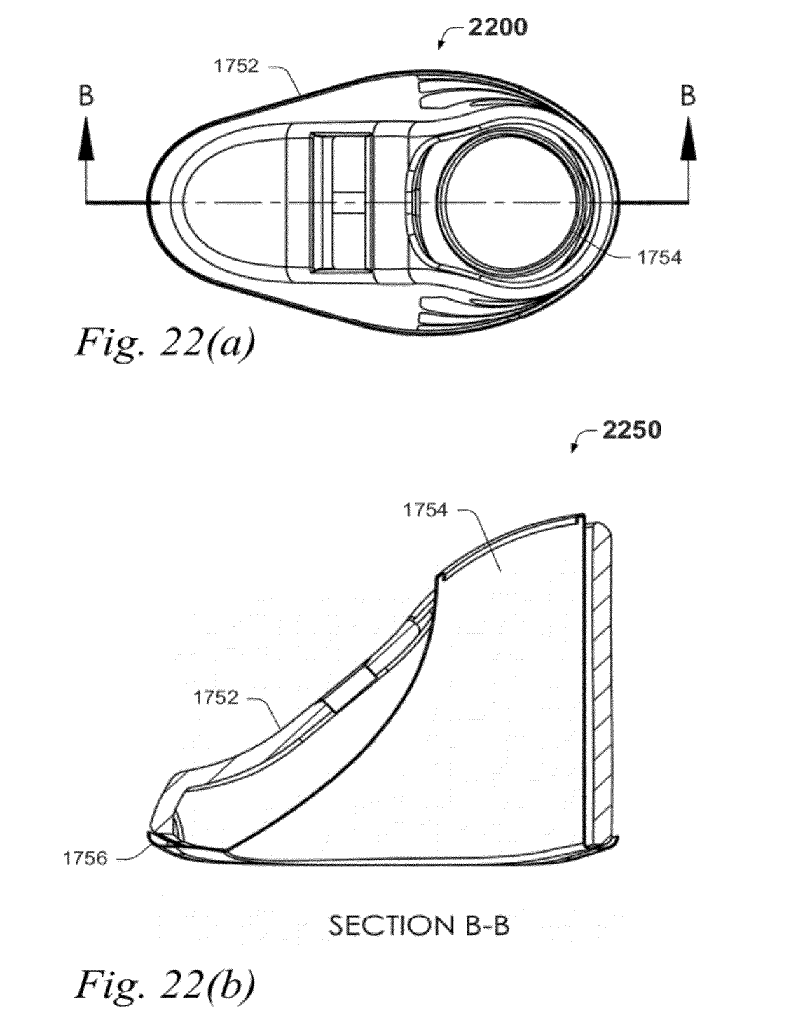

Referring to FIG. 22(a), a top plan view 2200 of one embodiment of a respiratory rate detection unit 1752 is presented.

Referring to FIG. 22(b), a right side cross sectional view 2250 of one embodiment of a respiratory rate detection unit 1752 is presented. The cross section is taken along section line B-B depicted in FIG. 22(a) with the front portion removed so the interior cavity of the respiratory rate detection unit can seen. The removable liner 1754 is disposed within the housing of the respiratory rate detection unit 1752. In one embodiment, the removable liner 1754 is removably attached to the respiratory rate detection unit 1752 device housing by a lip 1756 on the removable liner 1754, which interfaces with the device housing. In one embodiment, the removable liner 1754 extends beyond the top of the respiratory rate detection unit 1752. The sensor and other internal components have been omitted to better illustrate the structure of the device housing.

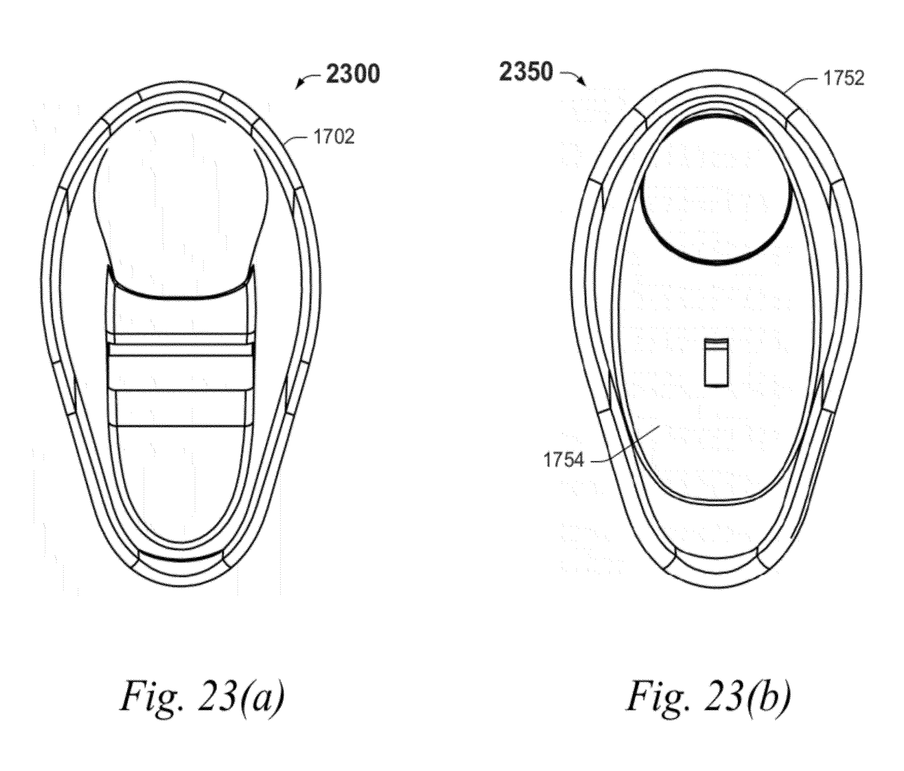

Referring to FIG. 23(a), a bottom plan view 2300 of one embodiment of a respiratory rate detection unit 1702 is presented.

Referring to FIG. 23(b), a bottom plan view 2350 of one embodiment of a respiratory rate detection unit 1752 with a liner 1754 is presented.

While the invention is described through the above-described exemplary embodiments, it will be understood by those of ordinary skill in the art that modifications to, and variations of, the illustrated embodiments may be made without departing from the inventive concepts disclosed herein. For example, although some aspects of making and using Applicants' respiratory rate detection system have been described with reference to a series of steps, those skilled in the art should readily appreciate that functions, operations, decisions, etc., of all or a portion of each block, or a combination of blocks, of the series of steps may be combined, separated into separate operations or performed in other orders. Moreover, while the embodiments are described in connection with various illustrative data structures, one skilled in the art will recognize that the respiratory rate detection system be embodied using a variety of dimensions. Furthermore, disclosed aspects, or portions of these aspects, may be combined in ways not listed above. Accordingly, the invention should not be viewed as being limited to the disclosed embodiment(s).

The present invention may be embodied in other specific forms without departing from its spirit or essential characteristics. The described implementations are to be considered in all respects only as illustrative and not restrictive. The scope of the invention should, therefore, be determined not with reference to the above description, but instead should be determined with reference to the pending claims along with their full scope or equivalents, and all changes which come within the meaning and range of equivalency of the claims are to be embraced within their full scope.

What is claimed is:

1. A respiration rate measurement device, comprising:
a tubular housing configured to be disposed over a nose and mouth on a face of a subject, wherein said tubular housing comprises:
a proximal end configured to communicate with said nose and mouth of said subject and receive a transient pressure event from said nose and mouth; and
a distal end that opens to ambient atmosphere, wherein the tubular housing is configured to guide a flow of air, generated from said transient pressure event, between said proximal end and said distal end; and
a sensor disposed within said tubular housing, wherein said sensor is configured to detect a respiration event by monitoring the flow of air within said tubular housing.

2. The respiration rate measurement device of claim 1, further comprising:
a cavity integrally formed within said tubular housing;
a battery disposed within said cavity; and
a control unit comprising a processor and a computer readable medium comprising computer readable program code disposed therein, wherein said control unit is configured to calculate a respiration rate.

3. The respiration rate measurement device of claim 2, further comprising:
a display connected to said tubular housing, wherein said display is configured to display said respiration rate.

4. The respiration rate measurement device of claim 2, wherein:
the control unit comprises a wireless communication unit; and
said wireless communication unit configured to transmit said respiration rate to an external device.

5. The respiration rate measurement device of claim 4, wherein said external device is configured to display said respiration rate.

6. The respiration rate measurement device of claim 1, further comprising:
a liner having a proximal end and a distal end, wherein:
said liner is disposed within said tubular housing and removably attached to said tubular housing;
said liner lines an interior surface of said tubular housing; and
said proximal end of said liner covers said proximal end of said tubular housing such that when said tubular housing is disposed over said nose and mouth of said subject, said liner is disposed between said tubular housing and said face of said subject, thereby preventing contact between said tubular housing and said face of said subject.

7. The respiration rate measurement device of claim 6, wherein said distal end of said liner extends beyond the distal end of said tubular housing.

8. The respiration rate measurement device of claim 7, wherein:
said interior surface of said tubular housing defines a first internal volume;
an interior surface of said liner defines a second internal volume; and
said second internal volume is between about 20 percent to about 95 percent that of said first internal volume.

9. The respiration rate measurement device of claim 6, wherein said sensor is non-removably integrated with said liner.

10. The respiration rate measurement device of claim 6, further comprising an opening formed in a side of said liner, wherein said sensor extends through said opening.

11. The respiration rate measurement device of claim 6, further comprising a strap to secure said tubular housing to said face of said subject.

12. The respiration rate measurement device of claim 1, wherein said sensor comprises a thermal anemometer.

13. The respiration rate measurement device of claim 1, wherein said sensor comprises a component selected from the group consisting of a microphone, an optical detector, a contact panel, a moisture sensor, and a carbon dioxide sensor.

14. A respiratory rate detection system comprising:
a tubular housing configured to be disposed over a nose and mouth on a face of a subject, wherein said tubular housing comprises:
a proximal end configured to communicate with said nose and mouth of said subject and receive a transient pressure event from said nose and mouth; and
a distal end that opens to ambient atmosphere, wherein said tubular housing is configured to guide a flow of air, generated from said transient pressure event, between said proximal end and said distal end;
a sensor disposed within said housing, wherein said sensor is configured to detect a respiration event by monitoring the flow of air within said tubular housing;
a processor and a computer readable medium comprising computer readable program code disposed therein to determine a respiration rate based on a plurality of detected events registered by said sensor; and
said computer readable program code comprising a series of computer readable program steps to effect:
initiating a timer to trigger at a predetermined time interval;
receiving respiration event data from said sensor corresponding to said plurality of respiration events detected by said sensor;
filtering said respiration event data to remove background noise and to identify at least one individual respiration cycle;
incrementing a value of a breath count variable for each said individual respiration cycle; and
upon said triggering of said timer, calculating said respiration rate.

15. The respiratory rate detection system of claim 14, wherein said calculating said respiration rate comprises:
determining a number of predetermined time intervals per minute; and
dividing the value of the breath count variable by the number of predetermined time intervals per minute.

16. The respiratory rate detection system of claim 15, wherein said predetermined time interval is 15 seconds and said number of predetermined time intervals per minute is 4.

17. The respiratory rate detection system of claim 14, wherein said sensor comprises a thermal anemometer.

18. The respiration rate detection system of claim 14, wherein said sensor comprises a component selected from the group consisting of a microphone, an optical detector, a contact panel, a moisture sensor, and a carbon dioxide sensor.

19. The respiration rate detection system of claim 14, wherein said series of computer readable program steps further includes displaying on a digital display said respiration rate.

20. The respiration rate detection system of claim 14, wherein said series of computer readable program steps further includes transmitting said respiration rate to an external device.
